(19) United States
(12) Patent Application Publication
(10) Pub. No.: US 2019/0314574 A1
(43) Pub. Date: Oct. 17, 2019

(54) DEVICES AND METHODS FOR PERCUTANEOUS LUNG INTRATUMORAL THERAPY DELIVERY

(71) Applicant: ALCYONE LIFESCIENCES, INC., Lowell, MA (US)

(72) Inventors: Katelyn Perkins-Neaton, Reading, MA (US); Gregory Eberl, Acton, MA (US); Morgan Brophy, Boston, MA (US); Andrew East, Arlington, MA (US); PJ Anand, Lowell, MA (US); Deep Arjun Singh, Cambridge, MA (US); Loredana Guseila, Belmont, MA (US); Jon Freund, Woburn, MA (US); Derek Peter, Shirley, MA (US)

(21) Appl. No.: 16/286,707

(22) Filed: Feb. 27, 2019

Related U.S. Application Data
(60) Provisional application No. 62/657,019, filed on Apr. 13, 2018.

(51) Int. Cl. A61M 5/168; A61M 25/00

(52) U.S. Cl. CPC A61M 5/16813; A61M 25/0068; A61M 2037/003; A61M 25/001; A61M 2210/1039; A61M 25/0084

(57) ABSTRACT

Percutaneous therapy or drug delivery devices are described herein. The device can include one or multiple lumens inside a cannula or catheter body. The device can include features for reducing or preventing backflow or reflux of infusate along the device insertion track, such as one or more bullet noses, over tubes, and/or micro-tips. The device can be used in any of a variety of treatment methods, including to inject cancer therapy medicinal products directly into pulmonary tumors or tumors located in other regions of the body. The device can include features to keep the distal tip secure during patient respiration or during other patient movement, and can reduce the incidence of reflux during therapy delivery.

### RELATED APPLICATION(S)

This application claims the benefit of U.S. Provisional Patent Application No. 62/657,019, filed on Apr. 13, 2018, the entire contents of which are incorporated herein by reference.

### FIELD

Devices and methods for therapy delivery are described herein, e.g., for percutaneous lung intratumoral therapy delivery.

### BACKGROUND

There are many instances in which it may be desirable to deliver a drug to a patient. The term “drug” as used herein refers to any functional agent that can be delivered to a human or animal subject, including hormones, stem cells, gene therapies, chemicals, compounds, small and large molecules, dyes, antibodies, viruses, therapeutic agents, etc.

There is a continual need for improved drug delivery systems and methods.

### SUMMARY

Percutaneous therapy or drug delivery devices are described herein. The device can include one or multiple lumens inside a cannula or catheter body. The device can include features for reducing or preventing backflow or reflux of infusate along the device insertion track, such as one or more bullet noses, over-tubes, and/or micro-tips. The device can be used in any of a variety of treatment methods, including to inject cancer therapy medicinal products directly into pulmonary tumors or tumors located in other regions of the body. The device can include features to keep the distal tip secure during patient respiration or during other patient movement, and can reduce the incidence of reflux during therapy delivery.

In some embodiments, the drug delivery device can include a distal tip having one or more fluid ports therein, an inner fluid lumen configured to convey fluid to the one or more fluid ports of the tip, and multiple bullet noses disposed in a spaced relationship along a length of the device proximal to the distal tip. The bullet noses can be configured to limit or prevent backflow of infusate along an exterior of the device. In certain embodiments, one or more of the bullet noses have a conical, curved, or tapered exterior surface. The bullet noses can engage surrounding tissue to anchor the device.

In some embodiments, the drug delivery device can further include means for anchoring the distal tip to target tissue of a patient to prevent movement of the distal tip relative to the target tissue during patient movement. The target tissue of a patient can include a tumor. The patient movement can include respiration. The means for anchoring can be separate from the plurality of bullet noses. In certain embodiments, the means for anchoring can include one or more splines deployable from an exterior of the device to engage surrounding tissue. In certain embodiments, the means for anchoring can include one or more balloons deployable from an exterior of the device to engage surrounding tissue.

In some embodiments, the device can include one or more over-tubes disposed over the distal tip to define a tissue-receiving space. The tissue can be received within the tissue-receiving space to limit or prevent backflow of infusate along an exterior of the device.

### BRIEF DESCRIPTION OF THE DRAWINGS

FIG. 1 is a perspective view of one exemplary embodiment of a CED device;

FIG. 2 is a cross-sectional view of the device of FIG. 1, taken in a plane normal to the longitudinal axis of the device;

FIG. 3 is a schematic view of a fluid delivery system that includes the device of FIG. 1;

FIG. 4 is a schematic view of the device of FIG. 1 inserted into tissue;

FIG. 5 is a perspective view of another exemplary embodiment of a CED device;

FIG. 6A is a plan view of another exemplary embodiment of a CED device;

FIG. 6B is a plan view of another exemplary embodiment of a CED device;

FIG. 6C is a plan view of another exemplary embodiment of a CED device;

FIG. 7 is a perspective view of another exemplary embodiment of a CED device;

FIG. 8 is another perspective view of the CED device of FIG. 7;

FIG. 9 is a perspective view of the CED device of FIG. 7 with a depth stop and tip protector;

FIG. 10 is a plan view of the CED device of FIG. 7 with a length of extension tubing;

FIG. 11 is a perspective view of a micro-tip of the CED device of FIG. 7;

FIGS. 12A, 12B, 12C, and 12D are schematic illustrations of one exemplary embodiment of an anchoring feature incorporated into a drug delivery device;

FIGS. 13A and 13B are schematic illustrations of one exemplary embodiment of an anchoring feature incorporated into a drug delivery device;

FIG. 14 is a schematic illustration of one exemplary embodiment of an anchoring feature incorporated into a drug delivery device;

FIGS. 15A and 15B are schematic illustrations of exemplary embodiments of an anchoring feature incorporated into a drug delivery device;

FIGS. 16A and 16B are schematic illustrations of one exemplary embodiment of an anchoring feature incorporated into a drug delivery device;

FIG. 17 is a schematic illustration of one exemplary embodiment of an anchoring feature incorporated into a drug delivery device;

FIGS. 18A and 18B are schematic illustrations of one exemplary embodiment of an anchoring feature incorporated into a drug delivery device;

FIGS. 19A and 19B are schematic illustrations of exemplary embodiments of an anchoring feature incorporated into a drug delivery device;

FIGS. 20A, 20B, and 20C are schematic illustrations of exemplary embodiments of an anchoring feature incorporated into a drug delivery device;

FIGS. 21A, 21B, and 21C are schematic illustrations of exemplary embodiments of an anchoring feature incorporated into a drug delivery device;

FIGS. 22A, 22B, and 22C are schematic illustrations of exemplary embodiments of an anchoring feature incorporated into a drug delivery device;

FIG. 23 is a schematic illustration of one exemplary embodiment of an anchoring feature incorporated into a drug delivery device;

FIG. 24 is a schematic illustration of one exemplary embodiment of an anchoring feature incorporated into a drug delivery device;

FIGS. 25A and 25B are schematic illustrations of one exemplary embodiment of a drug delivery device;

FIGS. 26A and 26B are schematic illustrations of one exemplary embodiment of a drug delivery device;

FIGS. 27A, 27B, and 27C are schematic illustrations of one exemplary embodiment of a drug delivery device;

FIG. 28 is a schematic illustration of one exemplary embodiment of a drug delivery device;

FIG. 29 is a schematic illustration of one exemplary embodiment of a drug delivery device;

FIG. 30A is a schematic illustration of one exemplary embodiment of a drug delivery device;

FIG. 30B is a schematic illustration of one exemplary embodiment of a drug delivery device;

FIG. 31 is a schematic illustration of one exemplary embodiment of a drug delivery device;

FIGS. 32A-32H are schematic illustrations of exemplary embodiments of needle tip geometries; and

FIGS. 33A-33F are schematic illustrations of exemplary embodiments of needle tip geometries.

### DETAILED DESCRIPTION

Certain exemplary embodiments will now be described to provide an overall understanding of the principles of the structure, function, manufacture, and use of the methods, systems, and devices disclosed herein. One or more examples of these embodiments are illustrated in the accompanying drawings. Those skilled in the art will understand that the methods, systems, and devices specifically described herein and illustrated in the accompanying drawings are non-limiting exemplary embodiments. The features illustrated or described in connection with one exemplary embodiment may be combined with the features of other embodiments. Such modifications and variations are intended to be included within the scope of the present disclosure.

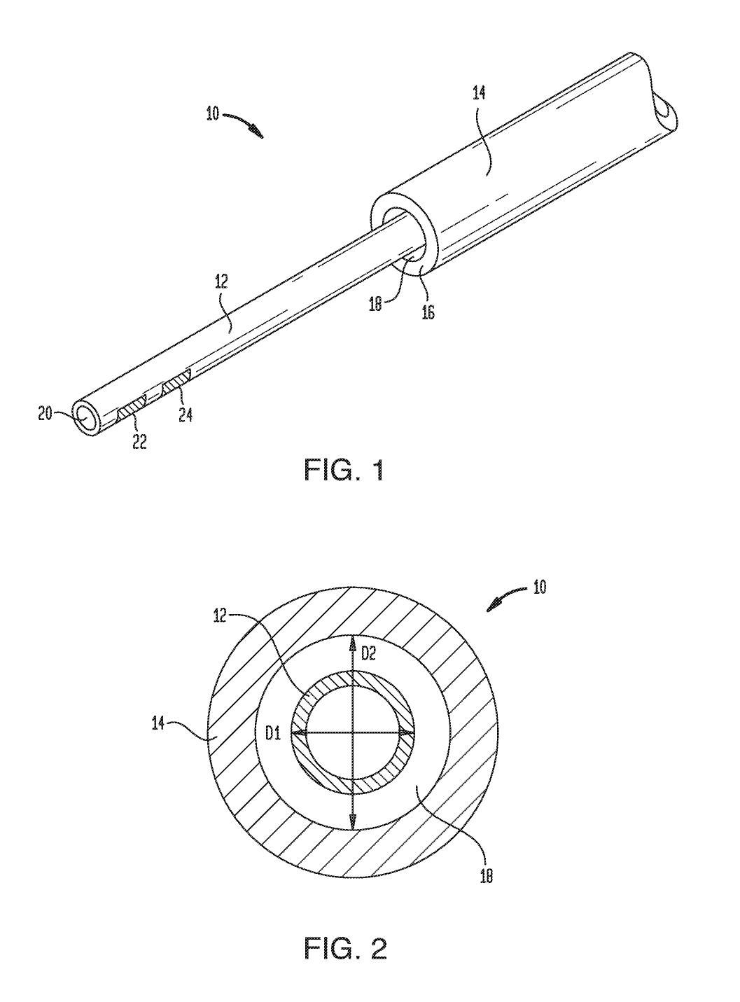

The devices disclosed herein can include any one or more of a micro-tip, one or more over-tubes, and one or more bullet nose features to reduce or prevent reflux. Exemplary micro-tip, over-tube, and bullet nose features are described in U.S. Pat. No. 8,992,458 entitled SYSTEMS AND METHODS FOR REDUCING OR PREVENTING BACKFLOW IN A DELIVERY SYSTEM, the entire contents of which are incorporated herein by reference, and FIGS. 1-11 of which are filed as same-numbered figures hereof and the corresponding description of which is reproduced below.

FIG. 1 illustrates one exemplary embodiment of a CED device 10. The device 10 generally includes a fluid conduit 12 and an outer sheath 14. The outer sheath 14 can be disposed coaxially over the fluid conduit 12 such that the fluid conduit 12 extends out of a distal end 16 of the outer sheath 14. The fluid conduit 12 and the outer sheath 14 can be sized and dimensioned such that a tissue-receiving space 18 is formed between an exterior surface of the fluid conduit 12 and an interior surface of the distal end 16 of the outer sheath 14.

The fluid conduit 12 can define one or more fluid lumens that extend generally parallel to the central longitudinal axis of the device 10. The fluid conduit 12 can include a fluid inlet port (not shown in FIG. 1) and a fluid outlet port 20. While a single fluid outlet port 20 is shown in the illustrated embodiment, it will be appreciated that the device can include a plurality of fluid outlet ports, as well as a plurality of fluid inlet ports and a plurality of fluid lumens extending therebetween. The fluid inlet port can be positioned at a proximal end of the device 10, and can allow the fluid conduit 12 to be placed in fluid communication with a fluid reservoir, e.g., via one or more catheters, pumps, meters, valves, or other suitable control devices. Such control devices can be used to regulate the pressure at which fluid is supplied to the device 10, or the rate or volume of fluid that is supplied to the device 10.

Fluid supplied to the conduit 12 though the fluid inlet port can be directed through one or more inner lumens of the conduit 12 and released through the one or more fluid outlet ports 20. The fluid outlet ports 20 can be sized, shaped, and/or positioned to control various release parameters of the fluid. For example, the fluid outlet ports 20 can be configured to control the direction in which fluid is released from the device 10, the distribution of the fluid within the target tissue, and the velocity or pressure at which the fluid is released. In exemplary embodiments, the size of the fluid outlet ports can progressively increase towards the distal end of the device 10, which can advantageously compensate for pressure loss that occurs along the length of the device such that fluid is released from each of the plurality of fluid outlet ports at substantially the same pressure. The fluid outlet ports can also be positioned at various points around the circumference of the fluid conduit 12 or can be shaped to control the release direction of the fluid.

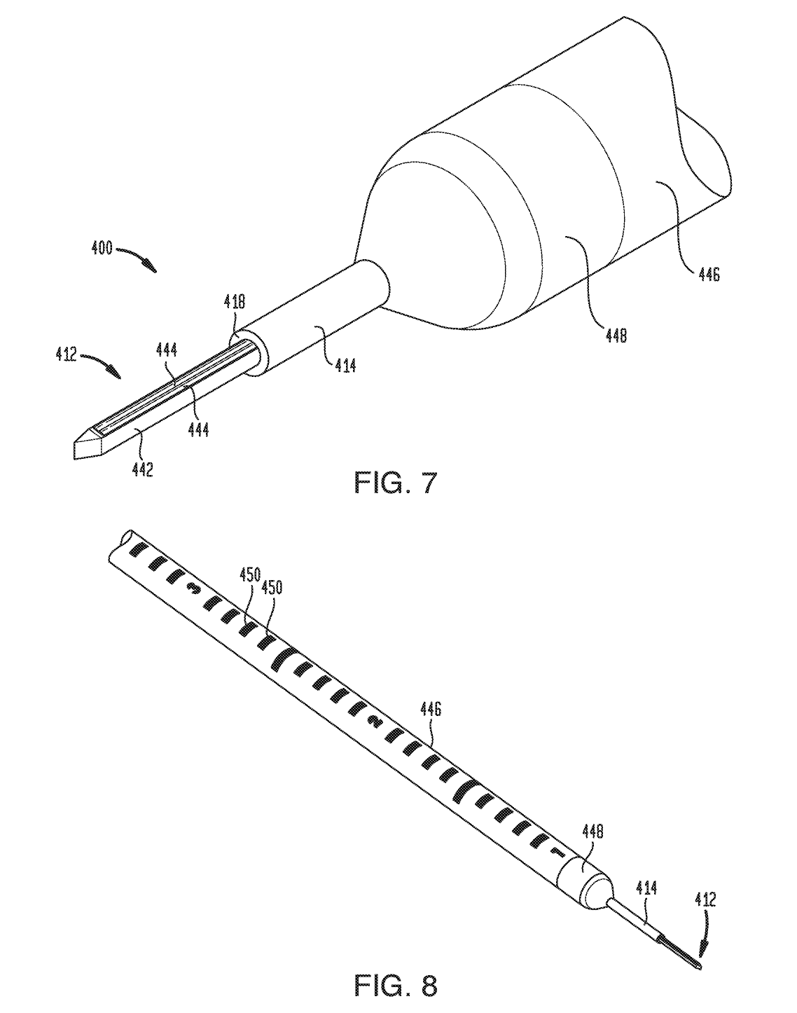

The fluid conduit 12 and/or the outer sheath 14 can have circular outside cross-sections, which can advantageously allow the device 10 to rotate within the tissue without causing trauma or forming large gaps between the exterior of the device and the surrounding tissue that might increase backflow. The fluid conduit 12 can also be flexible to allow it to move with the tissue in which it is inserted. While a generally-cylindrical fluid conduit 12 is shown, the fluid conduit 12 can also have a non-cylindrical or polygonal cross-section. For example, as described below with respect to FIG. 7, the fluid conduit 12 can be a microfabricated tip that includes a substrate having a square or rectangular cross-section with one or more fluid channels disposed thereon. The interior of the outer sheath 14 can be shaped to substantially correspond to the cross-section of the fluid conduit 12. Alternatively, the outer sheath 14 can have an interior cross-sectional shape that differs from the exterior cross-sectional shape of the fluid conduit 12. For example, the outer sheath 14 can have a substantially cylindrical interior cross-sectional shape at its distal end, while the fluid conduit 12 can have a substantially square or rectangular exterior cross-sectional shape, thereby defining the tissue-receiving space 18 between the exterior of the fluid conduit 12 and the interior of the outer sheath 14.

As noted above, the outer sheath 14 can be disposed coaxially over the fluid conduit 12 such that the fluid conduit 12 extends out of the distal end 16 of the outer sheath 14. A clearance space between the exterior surface of the fluid conduit 12 and the interior surface of the sheath 14 can define the tissue-receiving space 18. For example, as shown in FIG. 2, the fluid conduit 12 can have an outside diameter D1 that is less than an inside diameter D2 of the outer sheath 14. The degree to which the diameter D2 exceeds the diameter D1 can dictate the amount of tissue that is compressed into or pinched by the tissue-receiving space 18.

In some embodiments, an adhesive or other filler can be disposed between the fluid conduit 12 and the sheath 14 to hold the fluid conduit in a fixed longitudinal position relative to the sheath and to maintain the fluid conduit in the center of the sheath (e.g., such that the tissue-receiving space 18 has a uniform width about the circumference of the fluid conduit). For example, the tissue-receiving space 18 can extend proximally a first distance from the distal end 16 of the sheath 14, after which point the clearance space between the fluid conduit 12 and the sheath 14 can be filled. In some embodiments, the sheath 14 can have a stepped, tapered, or other similarly-shaped interior such that a clearance space exists along a distal portion of the sheath 14 and no clearance space exists along a proximal portion of the sheath 14.

In exemplary embodiments, the inside diameter of the distal end 16 of the outer sheath 14 can be about 1 μm to about 1000 μm, about 1 μm to about 500 μm, about 1 μm to about 200 μm, or about 1 μm to about 20 μm greater than the outside diameter of the fluid conduit 12. In exemplary embodiments, the inside diameter of the distal end 16 of the outer sheath 14 can be about 5 percent to about 500 percent, about 5 percent to about 250 percent, about 10 percent to about 100 percent, or about 10 percent to about 20 percent greater than the outside diameter of the fluid conduit 12. In exemplary embodiments, the diameter D1 can be about 50 μm to about 2000 μm, about 50 μm to about 1000 μm, or about 50 μm to about 200 μm. In exemplary embodiments, diameter D2 can be about 51 μm to about 5000 μm, about 55 μm to about 1000 μm, or about 55 μm to about 200 μm. The tissue-receiving space 18 can extend along the entire length of the outer sheath 14, or along only a portion of the outer sheath (e.g., along about 1 mm to about 100 mm, about 1 mm to about 50 mm, or about 1 mm to about 10 mm of the distal-most portion of the outer sheath).

The fluid conduit 12 and the outer sheath 14 can be formed from any of a variety of materials, including parylene compositions, silastic compositions, polyurethane compositions, PTFE compositions, silicone compositions, and so forth.

In some embodiments, the device 10 can be mounted on a support scaffold (not shown) to provide structural rigidity to the device and facilitate insertion into the target tissue. Exemplary support scaffolds are illustrated and described in U.S. Publication No. 2013/0035560, filed on Aug. 1, 2012, entitled “MULTI-DIRECTIONAL MICROFLUIDIC DRUG DELIVERY DEVICE,” the entire contents of which are incorporated herein by reference. To assist with tissue penetration and navigation, the distal end of the fluid conduit 12 and/or the distal end of the scaffold can be tapered, pointed, and/or sharpened. In some embodiments, the fluid conduit 12 and/or the scaffold can be provided with a rounded atraumatic tip so as to facilitate insertion through tissue without causing trauma to the tissue. The support scaffold can be rigid or semi-rigid and can be formed from a degradable thermoplastic polymer, for example, a degradable thermoplastic polyester or a degradable thermoplastic polycarbonate. In some embodiments, the support scaffold can be formed from poly(lactic-co-glycolic acid) (PLGA) and can be configured to biodegrade within the target tissue. This can advantageously eliminate the need to remove the support scaffold once the device 10 is positioned within target tissue, thereby avoiding the potential to disrupt the positioning of the fluid conduit 12. Any of a variety of other materials can also be used to form the support scaffold, including silicon or various ceramics, metals, and plastics known in the art. The scaffold can have a width of approximately 100 μm to approximately 200 μm and can have a length that varies depending on the target tissue (e.g., depending on the depth at which the target tissue is situated). In one embodiment, the scaffold is between 2 cm and 3 cm long. A variety of techniques can be used to couple the fluid conduit 12 and/or the outer sheath 14 to the support scaffold, such as surface tension from a water drop, adhesives, and/or a biocompatible petroleum jelly.

Any of the fluid conduit 12, the outer sheath 14, and/or the support scaffold can contain or can be impregnated with a quantity of a drug. Alternatively, or in addition, a surface of these components can be coated with a drug. Exemplary drugs include anti-inflammatory components, drug permeability-increasing components, delayed-release coatings, and the like. In some embodiments, one or more components of the device 10 can be coated or impregnated with a corticosteroid such as dexamethasone which can prevent swelling around the injection site and disruptions to the fluid delivery pattern that can result from such swelling.

The device 10 can also include one or more sensors 22 mounted in or on the fluid conduit 12, the sheath 14, or the scaffold. The sensors 22 can include temperature sensors, pH sensors, pressure sensors, oxygen sensors, tension sensors, interrogatable sensors, glutamate sensors, ion concentration sensors, carbon dioxide sensors, lactate sensors, neurotransmitter sensors, or any of a variety of other sensor types, and can provide feedback to a control circuit which can in turn regulate the delivery of fluid through the device 10 based on one or more sensed parameters. One or more electrodes 24 can also be provided in or on the fluid conduit 12, the sheath 14, or the scaffold, which can be used to deliver electrical energy to target tissue, e.g., to stimulate the target tissue or to ablate the target tissue. In one embodiment, electrical energy is delivered through an electrode 24 while a drug is simultaneously delivered through the fluid conduit 12.

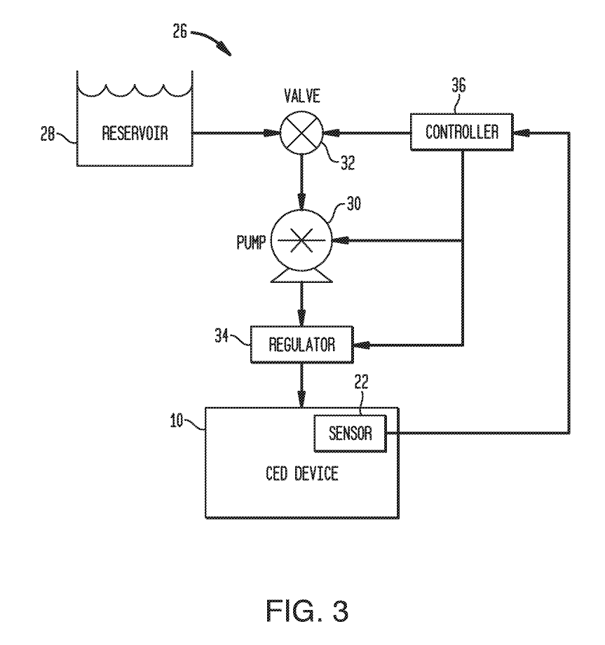

FIG. 3 is a schematic illustration of a drug delivery system 26 that includes the device 10. The system 26 includes a reservoir 28 of a drug-containing fluid that is coupled to a pump 30 via a control valve 32. When the control valve 32 is opened, fluid in the reservoir 28 is supplied under pressure by the pump 30 to a pressure regulator 34, which can adjust a pressure at which the fluid is supplied to the device 10. The control valve 32, pump 30, and regulator 34 can be operatively coupled to a controller 36 which can include a microprocessor and a memory and can be configured to execute a drug-delivery control program stored in a non-transitory computer-readable storage medium. The controller 36 can be configured to open or close the valve 32, to turn the pump 30 on or off, to change an output pressure of the pump 30, and/or to adjust a pressure set point of the regulator 34. The controller 36 can also receive information indicative of a sensed parameter via a feedback loop that includes one or more sensors 22 mounted in or on the device 10. Thus, in response to feedback from one or more sensors 22 implanted with the device 10, the controller 36 can start or stop the flow of fluid to the device 10, increase or decrease the pressure at which fluid is supplied to the device 10, etc. In one embodiment, the device 10 includes a pressure sensor 22 that measures a fluid pressure in the vicinity of the device 10 and the controller 36 is configured to maintain the fluid supply pressure at a substantially constant level based on feedback from the pressure sensor 22.

The device 10 can be used for CED of drugs to treat disorders of the brain, spine, ears, neural tissue, or other parts of a human or animal body. When used in the brain, the device 10 can circumvent the blood-brain barrier (BBB) by infusing drugs under positive pressure directly into tissue. The device 10 can provide a number of advantages, such as 1) a smaller cross-sectional area compared with conventional needles used in CED; 2) less disturbance to tissue when inserted into the brain than conventional needles; 3) the reduction or elimination of backflow or reflux along the outside of the inserted part, which in turn, permits higher rates of drug delivery in the device 10 compared with conventional needles; 4) minimal or no occlusion of the fluid delivery conduit 12 during insertion into the brain; 5) multiple lumens can be provided through the fluid conduit 12, each conducting a distinct fluid (drug), which allows simultaneous, sequential, or programmed delivery of multiple agents; 6) the device 10 has the potential to serve simultaneously as a drug delivery system and as a sensor-equipped probe to measure local tissue characteristics such as, but not limited to, pressure, pH, ion-specific concentrations, location, and other parameters; and 7) the device 10 allows for directional control of the drug release pattern.

In use, as described further below, the device 10 can be functionally attached to the distal end of a long, thin insertion vehicle such as a cannula or a needle in or on which a fluid attachment can be made to the fluid inlet port of the device's fluid conduit 12. This can be especially advantageous in applications involving penetration of relatively thick tissue, e.g., insertion through a human skull.

In addition to delivering a drug-containing fluid, the device 10 can also be used to deliver enzymes or other materials to modify tissue permeability and improve drug distribution in the targeted tissue. For example, penetration of drug-containing nanoparticles into brain tissue can be enhanced by enzymatic digestion of at least one brain extracellular matrix component and intracranial infusion of the nanoparticle into the brain tissue. In another embodiment, at least one enzyme can be immobilized to a surface of the nanoparticle during the step of enzymatic digestion. The device 10 can provide the ability to deliver enzymatic and/or other materials that can, e.g., modify the drug delivery site, and therapeutic materials, in virtually any order, sequencing, and/or timing without the need to use different delivery devices and the potential complications involved in doing so.

The device 10 can also be used to biopsy tissue, for example by passing a stylet or a grasping tool through the fluid conduit 12 to a target site and then withdrawing the stylet or grasping tool from the target site with a biopsy specimen therein. In some embodiments, the fluid conduit 12 can have a larger-diameter lumen extending therethrough for biopsy purposes, with smaller fluid lumens formed therearound.

The device 10 can be used to deliver a drug-containing fluid under positive pressure to a target tissue region. FIG. 4 illustrates an exemplary method for convection-enhanced delivery of a drug to target tissue 40 in a patient's brain. After appropriate site preparation and cleaning, a tissue opening can formed through the patient's scalp and skull to expose the brain tissue 40. Before or after forming the tissue opening, a pedestal can optionally be mounted to the patient to support the device 10 while it is inserted, which can be particularly useful in long-term implantations.

The device 10 can optionally be coupled to a cannula (not shown) with a microfabricated interface for mating with the device 10. Any of a variety of cannulas can be used, including standard cannulas configured to mate to a stereotactic frame in guided surgery. In some embodiments, the cannula can include a flexible catheter suitable for extended (e.g., 30 day) implantation. The catheter can be about 15 cm long and about 2 cm in diameter. The cannula can include a tubing portion that is approximately 6 feet in length with connectors for fluid and biosensor interface at the proximal end.

The device 10 can be advanced through the tissue opening and into the brain tissue 40. As shown, the tissue-receiving space 18 can be configured to compress or pinch tissue received therein as the device 10 is advanced through the tissue 40. Tissue compressed by the tissue-receiving space 18 can form a seal that reduces proximal backflow of fluid ejected from the outlet 20 of the fluid conduit 12 beyond the tissue-receiving space 18. In particular, as fluid ejected from the outlet 20 of the fluid conduit 12 flows back proximally between the exterior surface of the fluid conduit 12 and the surrounding tissue 40, it encounters a shoulder of tissue 38 that is compressed into the tissue-receiving space 18. Compression of the tissue 38 against the walls of the tissue-receiving space 18 forms a seal that resists flow of the fluid further in the proximal direction, thereby reducing or preventing undesirable backflow of injected fluid away from the target region of the tissue.

As explained above, the device 10 can include a support scaffold to facilitate penetration through the brain tissue towards the target region. One or more radiopaque markers can be included in the device 10 to permit radiographic imaging (e.g., to confirm proper placement of the device 10 within or in proximity to the target tissue). In embodiments in which a degradable scaffold is used, the scaffold can degrade shortly after insertion to leave behind only the fluid conduit 12 and outer sheath 14. In some embodiments, the fluid conduit 12 and/or the sheath 14 can be flexible to permit the device 10 to move with the brain tissue 40 if the brain tissue 40 shifts within the skull. This can advantageously prevent localized deformation of brain tissue adjacent to the device 10 that might otherwise occur with a rigid device. Such deformation can lead to backflow of the pressurized fluid along the surface of the device, undesirably preventing the fluid from reaching the target tissue.

Once the device 10 is positioned within or adjacent to the target tissue, injected media (e.g., a drug-containing fluid) can be supplied under positive pressure to the device 10 through its fluid inlet port(s). The injected media then flows through the fluid conduit 12 and is expelled under pressure from the outlet port(s) 20 in the target region of tissue. The delivery profile can be adjusted by varying parameters such as outlet port size, outlet port shape, fluid conduit size, fluid conduit shape, fluid supply pressure, fluid velocity, etc. In some embodiments, the device 10 can be configured to deliver fluid at a flow rate between about 5 μl per minute and about 20 μl per minute. In some embodiments, the device 10 can be configured to deliver 50-100 μl per minute per channel, and each channel can be configured to support greater than 100 psi of pressure.

In some embodiments, prior to injecting the drug-containing fluid, a gel or other material can be injected through the device 10 to augment the tissue seal. For example, a sealing gel can be injected through the device 10 and allowed to flow back along the exterior of the device, filling and sealing any voids that may exist between the device and the surrounding tissue, particularly within the tissue-receiving recess 18. Exemplary sealing materials include cyanoacrylate, protein glues, tissue sealants, coagulative glues (e.g., fibrin/thrombin/protein based coagulative glues), and materials such as those disclosed in U.S. Publication No. 2005/0277862, filed on Jun. 9, 2004, entitled “SPLITABLE TIP CATHETER WITH BIORESORBABLE ADHESIVE,” the entire contents of which are incorporated herein by reference.

It will be appreciated from the foregoing that the methods and devices disclosed herein can provide convection-enhanced delivery of functional agents directly to target tissue within a patient with little or no backflow. This convection-enhanced delivery can be used to treat a broad spectrum of diseases, conditions, traumas, ailments, etc. The term “drug” as used herein refers to any functional agent that can be delivered to a human or animal patient, including hormones, stem cells, gene therapies, chemicals, compounds, small and large molecules, dyes, antibodies, viruses, therapeutic agents, etc.

In some embodiments, central-nervous-system (CNS) neoplasm can be treated by delivering an antibody (e.g., an anti-epidermal growth factor (EGF) receptor monoclonal antibody) or a nucleic acid construct (e.g., ribonucleic acid interference (RNAi) agents, antisense oligonucleotide, or an adenovirus, adeno-associated viral vector, or other viral vectors) to affected tissue. Epilepsy can be treated by delivering an anti-convulsive agent to a target region within the brain. Parkinson's disease can be treated by delivering a protein such as glial cell-derived neurotrophic factor (GDNF) to the brain. Huntington's disease can be treated by delivering a nucleic acid construct such as a ribonucleic acid interference (RNAi) agent or an antisense oligonucleotide to the brain. Neurotrophin can be delivered to the brain under positive pressure to treat stroke. A protein such as a lysosomal enzyme can be delivered to the brain to treat lysosomal storage disease. Alzheimer's disease can be treated by delivering anti-amyloids and/or nerve growth factor (NGF) under positive pressure to the brain. Amyotrophic lateral sclerosis can be treated by delivering a protein such as brain-derived neurotrophic factor (BDNF) or ciliary neurotrophic factor (CNTF) under positive pressure to the brain, spinal canal, or elsewhere in the central nervous system. Chronic brain injury can be treated by delivering a protein such as brain-derived neurotrophic factor (BDNF) and/or fibroblast growth factor (FGF) under positive pressure to the brain.

It will be appreciated that use of the devices disclosed herein and the various associated treatment methods is not limited to the brain of a patient. Rather, these methods and devices can be used to deliver a drug to any portion of a patient's body, including the spine. By way of further example, balance or hearing disorders can be treated by injecting a drug-containing fluid directly into a portion of a patient's ear. Any of a variety of drugs can be used to treat the ear, including human atonal gene. The methods and devices disclosed herein can also be used to deliver therapeutics (such as stem cells) to a fetus or to a patient in which the fetus is disposed. The methods and devices disclosed herein can be used to treat a cavernous malformation, for example by delivering one or more antiangiogenesis factors thereto.

Any of the various treatments described herein can further include delivering a cofactor to the target tissue, such as a corticosteroid impregnated in the device, a corticosteroid coated onto the device, and/or a propagation enhancing enzyme. In addition, any of the various treatments described herein can further include long-term implantation of the device (e.g., for several hours or days) to facilitate long-term treatments and therapies.

A number of variations on the device 10 are set forth below. Except as indicated, the structure and operation of these variations is identical to that of the device 10, and thus a detailed description is omitted here for the sake of brevity.

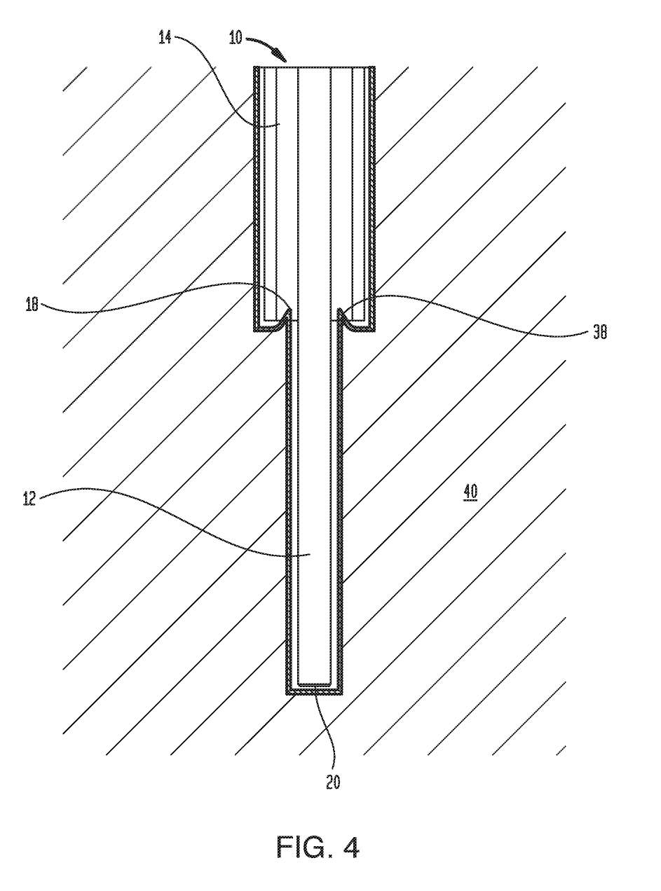

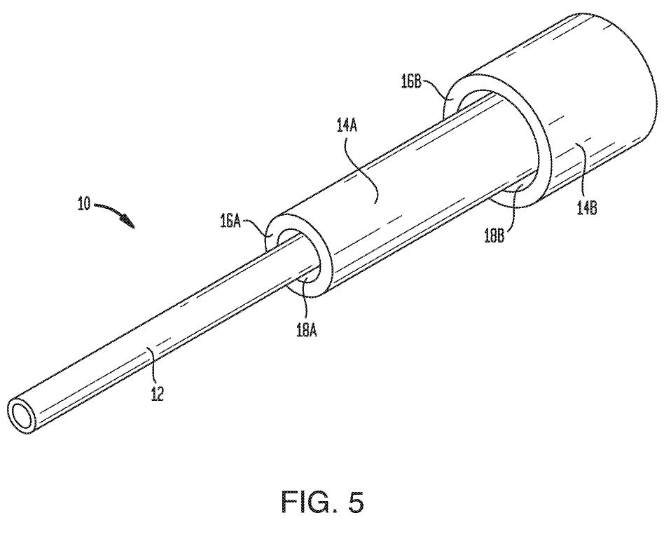

In some embodiments, the device 10 can include a plurality of tissue-receiving spaces 18. FIG. 5 illustrates an embodiment with a first tissue-receiving space 18A and a second tissue-receiving space 18B. As shown, a first outer sheath 14A is disposed over the fluid conduit 12 to define the first tissue-receiving space 18A. A second outer sheath 14B is disposed over the first outer sheath 14A to define the second tissue-receiving space 18B. Specifically, the second tissue-receiving space 18B is formed between an exterior surface of the first outer sheath 14A and an interior surface of the distal end 16B of the second outer sheath 14B. While two tissue-receiving spaces are shown, it will be appreciated that any number of tissue-receiving spaces can be provided (e.g., three, four, five, or more) by adding additional sheath layers. A single sheath layer can also be configured to provide multiple tissue-receiving spaces, for example by forming the sheath layer with one or more stepped regions, each stepped region defining a tissue-receiving space therein. Multi-stage devices such as that shown in FIG. 5 can provide additional sealing regions proximal to the distal-most, primary sealing region. The provision of these secondary, tertiary, etc. sealing regions can augment the primary seal or act as a backup in case the primary seal is compromised.

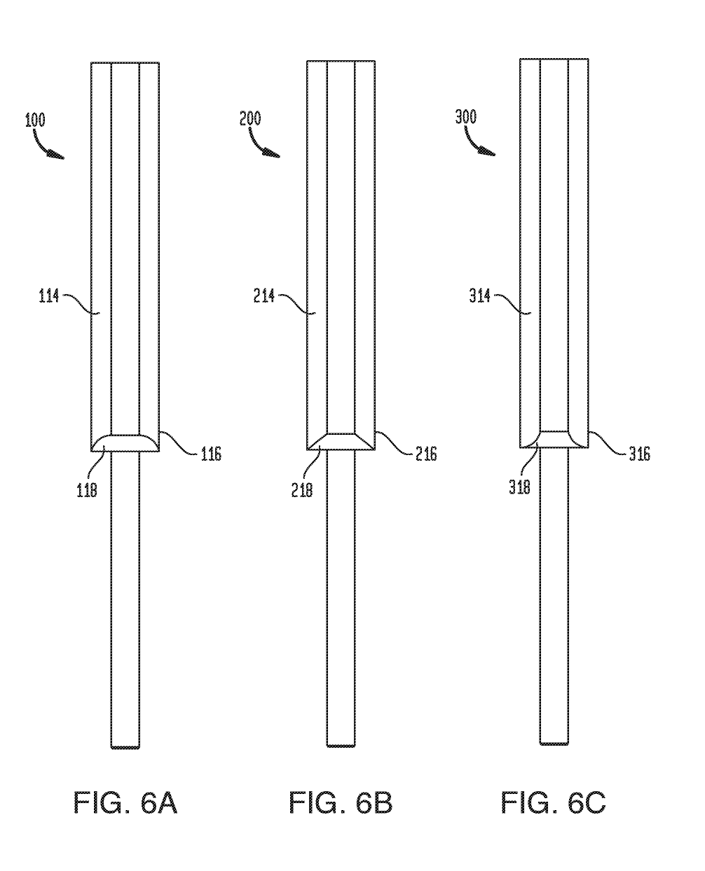

As shown in FIGS. 6A-6C, the internal wall of the distal end 16 of the outer sheath 14 can be shaped to alter the dimensions of the tissue-receiving space 18 and the type of seal provided when tissue is compressed therein. FIG. 6A illustrates a device 100 in which the interior surface of the distal end 116 of the sheath 114 has a concave curvature. FIG. 6B illustrates a device 200 in which the interior surface of the distal end 216 of the sheath 214 is conical. FIG. 6C illustrates a device 300 in which the interior surface of the distal end 316 of the sheath 314 has a convex curvature. These configurations can provide for a sharper leading edge at the periphery of the sheath as compared with the cylindrical tissue-receiving space 18 of the device 10, and can increase the amount of tissue compressed into or pinched/pinned by the tissue-receiving space, as well as the degree of compression. A more-robust seal can thus be obtained in some instances using the configurations of FIGS. 6A-6C. It should be noted, however, that even in the case of a cylindrical tissue-receiving space, the leading edge of the sheath can be sharpened to deflect tissue into the tissue-receiving space and thereby form a better seal. The size and shape of the tissue-receiving space can be selected based on a variety of parameters, including the type of tissue in which the device is to be inserted. In embodiments with a plurality of tissue-receiving spaces, each of the tissue receiving spaces can have the same configuration (e.g., all cylindrical, all conical, all convex, or all concave). Alternatively, one or more of the plurality of tissue-receiving spaces can have a different configuration. Thus, for example, one or more tissue-receiving spaces can be cylindrical while one or more other tissue receiving spaces are convex.

The tissue-receiving recesses of the devices disclosed herein can include various surface features or treatments to enhance the seal formed between the device and the surrounding tissue or gel. For example, the tissue-receiving recesses can be coated with a biocompatible adhesive or can have a textured surface to form a tighter seal with the tissue or gel.

FIG. 7 illustrates an exemplary embodiment of a CED device 400 that generally includes a fluid conduit in the form of a micro-tip 412 and an outer sheath 414. The micro-tip 412 includes a substrate 442, which can be formed from a variety of materials, including silicon. The substrate 442 can have any of a variety of cross-sectional shapes, including a square or rectangular cross-section as shown. One or more fluid channels 444 can be formed on the substrate 442. The fluid channels 444 can be formed from a variety of materials, including parylene. Additional details on the structure, operation, and manufacture of microfabricated tips such as that shown in FIG. 7 can be found in U.S. Publication No. 2013/0035560, filed on Aug. 1, 2012, entitled “MULTI-DIRECTIONAL MICROFLUIDIC DRUG DELIVERY DEVICE,” the entire contents of which are incorporated herein by reference.

The outer sheath 414 can be disposed coaxially over the micro-tip 412 so as to form a tissue-receiving space 418 therebetween. In some embodiments, the micro-tip 412 can have a substantially rectangular exterior cross-section and the outer sheath 414 can have a substantially cylindrical interior cross-section. In other embodiments, the micro-tip 412 and the outer sheath 414 can have corresponding cross-sectional shapes with a clearance space defined therebetween. The proximal end of the outer sheath 414 can be coupled to a catheter 446. The catheter 446 can be rigid or flexible, or can include rigid portions and flexible portions. A nose portion 448 (sometimes referred to herein as a “bullet nose” or a “bullet nose portion”) can be disposed between the outer sheath 414 and the catheter 446, or can be disposed over a junction between the outer sheath 414 and the catheter 446. As shown, the nose portion 448 can taper from a reduced distal diameter corresponding to the outside diameter of the sheath 414 to an enlarged proximal diameter corresponding to the outside diameter of the catheter 446. The tapered transition provided by the nose portion 448 can advantageously provide stress-relief as it can act as a smooth transition from the sheath 414 to the catheter body 446, avoiding any uneven stresses on the surrounding tissue that may create paths for fluid backflow. The nose portion 448 can be conically tapered, as shown, or can taper along a convex or concave curve. Various compound shapes can also be used that include conical portions, convex portions, and/or concave portions. The nose portion 448 can also be replaced with a blunt shoulder that extends perpendicular to the longitudinal axis of the device 400. Any of a variety of taper angles can be used for the nose portion 448. For example the nose portion 448 can taper at an angle in a range of about 10 degrees to about 90 degrees relative to the longitudinal axis of the device 400, in a range of about 20 degrees to about 70 degrees relative to the longitudinal axis of the device, and/or in a range of about 30 degrees to about 50 degrees relative to the longitudinal axis of the device. For example, the nose portion 446 can taper at an angle of approximately 33 degrees relative to the longitudinal axis of the device 400. In some embodiments, additional sheaths can be provided, e.g., as described above with respect to FIG. 5.

As shown in FIG. 8, the catheter 446 can include length markings or graduations 450 to indicate the insertion depth of the device 400. In some embodiments, the catheter 446 can be a straight rigid catheter sized and configured for acute stereotactic targeting. The catheter 446 can be formed from any of a variety of materials, including flexible materials, rigid materials, ceramics, plastics, polymeric materials, PEEK, polyurethane, etc. and combinations thereof. In an exemplary embodiment, the catheter 446 has length of about 10 cm to about 40 cm, e.g., about 25 cm. The catheter 446 can include one or more fluid lines extending therethrough. The fluid lines can be defined by the catheter body itself or can be defined by one or more inner sleeves or linings disposed within the catheter body. Any of a variety of materials can be used to form the inner sleeves or linings, such as flexible materials, rigid materials, polyimide, pebax, PEEK, polyurethane, silicone, fused silica tubing, etc. and combinations thereof.

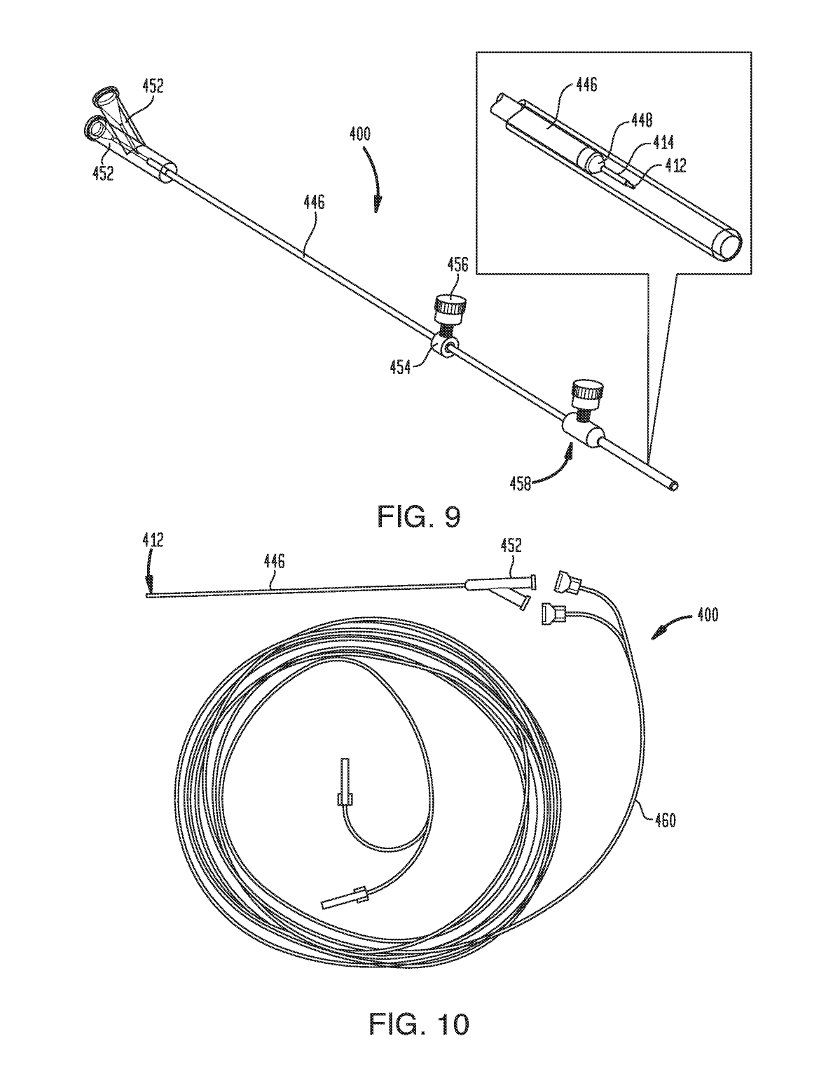

As shown in FIG. 9, one or more standard Luer or other connectors 452 can be coupled to the proximal end of the catheter 446 to facilitate connection with a fluid delivery system of the type shown in FIG. 3. In the illustrated embodiment, the system 400 includes two connectors 452, one for each of the two fluid channels formed in the catheter 446 and the micro-tip 412. It will be appreciated, however, that any number of fluid channels and corresponding proximal catheter connectors can be provided. The system 400 can also include a collar 454 disposed over the catheter 446 to act as a depth stop for setting the desired insertion depth and preventing over-insertion. The collar 454 can be longitudinally slidable with respect to the catheter 446 and can include a thumb screw 456 for engaging the catheter to secure the collar in a fixed longitudinal position with respect thereto. The system 400 can also include a tip protector 458 for preventing damage to the micro-tip 412 during insertion into stereotactic frame fixtures. Exemplary tip protectors are disclosed in U.S. Provisional Application No. 61/835,905, filed on Jun. 17, 2013, entitled “METHODS AND DEVICES FOR PROTECTING CATHETER TIPS,” the entire contents of which are incorporated herein by reference.

As shown in FIG. 10, the system 400 can include a length of extension tubing 460 to provide a fluid pathway between the proximal connectors 452 of the catheter 446 and a fluid delivery system of the type shown in FIG. 3. In the illustrated embodiment, dual-channel peel-away extension lines 460 are shown. In an exemplary method of using the system 400, an incision can be formed in a patient and the catheter 446 can be inserted through the incision and implanted in a target region of tissue (e.g., a region of the patient's brain or central nervous system). The catheter 446 can be left in the target region for minutes, hours, days, weeks, months, etc. In the case of a flexible catheter 446, the proximal end of the catheter can be tunneled under the patient's scalp with the proximal connectors 452 extending out from the incision. The catheter 446 can be inserted through a sheath to keep the catheter stiff and straight for stereotactic targeting. Alternatively, or in addition, a stylet can be inserted through the catheter to keep the catheter stiff and straight for stereotactic targeting. In some embodiments, the stylet can be inserted through an auxiliary lumen formed in the catheter such that the primary fluid delivery lumen(s) can be primed with fluid during catheter insertion. Thus, in the case of a catheter with first and second fluid lumens, a third lumen can be included for receiving the stylet.

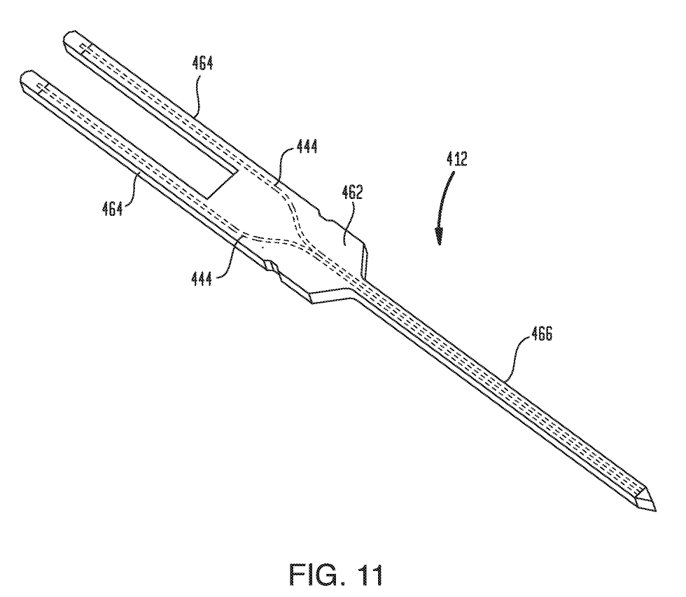

FIG. 11 is a close-up view of the exemplary micro-tip 412. As shown, the micro-tip 412 generally includes a central body portion 462 with first and second legs or tails 464 extending proximally therefrom and a tip portion 466 extending distally therefrom. First and second microfluidic channels 444 are formed in or on the micro-tip 412 such that they extend along the proximal legs 464, across the central body portion 462, and down the distal tip portion 466. The channels 444 can each include one or more fluid inlet ports (e.g., at the proximal end) and one or more fluid outlet ports (e.g., at the distal end). As noted above, additional details on the structure, operation, and manufacture of microfabricated tips such as that shown in FIG. 11 can be found in U.S. Publication No. 2013/0035560, filed on Aug. 1, 2012, entitled “MULTI-DIRECTIONAL MICROFLUIDIC DRUG DELIVERY DEVICE,” the entire contents of which are incorporated herein by reference.

The devices disclosed herein can include a single lumen or multiple independent lumens, e.g., discrete lumens for drug or therapy delivery and for delivery of imaging agents. The lumens can remain independent throughout their length, or can merge or be combined together at the distal tip of the device or at a location proximal to an outlet port of the device. The proximal end of the device can include clear markings or other identification of each unique lumen to assist the user in determining, for example, which lumen is to be used for imaging agents and which is to be used for therapy. The device can allow for an “aura” or “halo” method of visualizing infusion, e.g., as described in U.S. Publication No. 2016/0213312 entitled DRUG DELIVERY METHODS WITH TRACER, the entire contents of which are incorporated herein by reference.

The devices disclosed herein can include any of a variety of anchoring features to allow the distal tip or other portion of the device to remain in place at the delivery location during infusion to reduce movement of the device when the patient moves. For example, in the case of delivery into a lung tumor, the anchoring features can limit or prevent movement of the device relative to the tumor during patient respiration. The anchoring features can be selectively deployable in response to user input. For example, the device can include a proximal hub with a lever, handle, or other actuator for advancing or retracting the anchoring features to deploy or withdraw the anchoring features to or from surrounding tissue. The proximal end of the device can be easily connected to extension lines, syringes, pumps, or other delivery components to facilitate infusion and/or aspiration through the device. The devices disclosed herein can be used with the patient under jet ventilation to reduce respiratory motion and improve delivery of therapy.

The devices disclosed herein can include markings at various locations to signify features of the device. For example, the device can include length markings and/or a radiopaque feature near the distal tip to indicate the microtip or fluid port location.

The devices disclosed herein can be inserted through or mounted or attached to the distal end of a stiff or flexible catheter or cannula body. The device can be delivered, guided, and used with standard CT or ultrasound guidance. The device can have a 16 gauge or smaller cannula body size to prevent or reduce the risk of pneumothorax. The device, or the lumens or other component parts thereof, can be formed from any of a variety of materials. Exemplary materials can include fused silica, PEEK, polyurethanes, PTFEs, FEPs, LDPE, metal, plastic, silica, and combinations thereof. The devices disclosed herein can be used to deliver any of a variety of drugs, including Antisense oligonulceotides, Adeno Viruses, Gene therapy (AAVs and non-AAV) including gene editing and gene switching, Oncolytic immunotherapies, monoclonal and polyclonal antibodies, stereopure nucleic acids, small molecules, methotrexate, and the like.

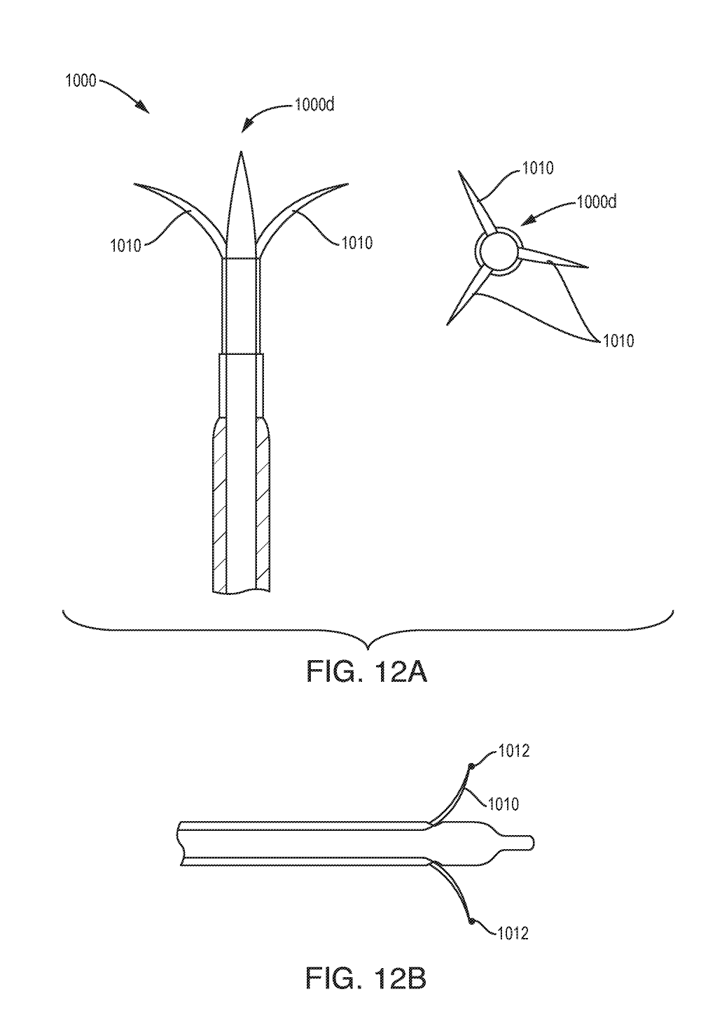

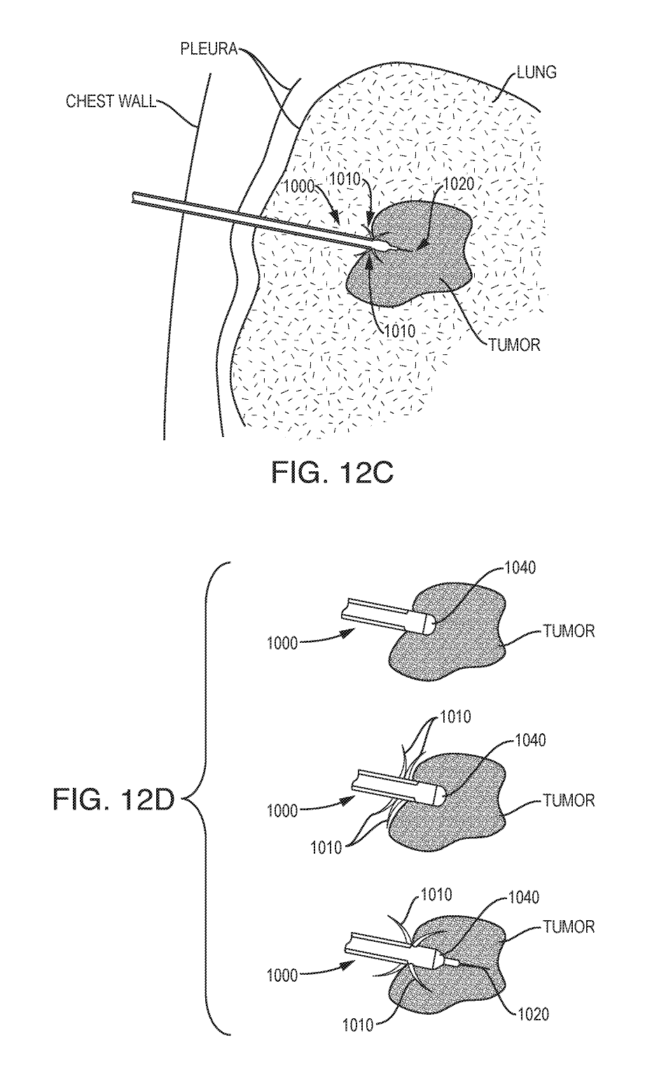

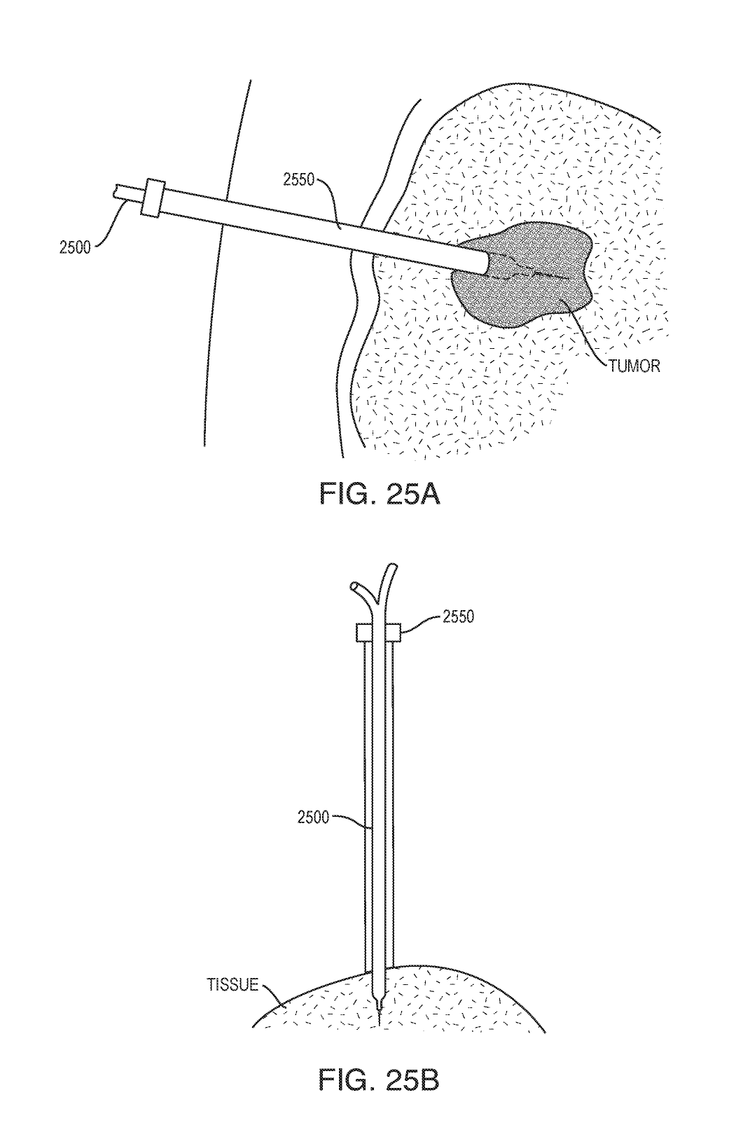

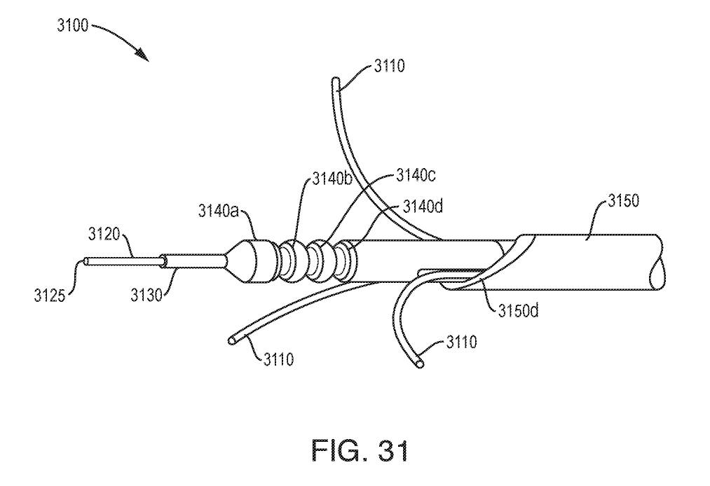

FIGS. 12A-24 illustrate exemplary anchoring features that can be incorporated singularly or in combination into the delivery devices described herein. FIGS. 25A-30B illustrate other system features that can be incorporated singularly or in combination into the delivery devices described herein. FIG. 31 illustrates an exemplary delivery device.

As shown in FIG. 12A, the device 1000 can include retractable spline anchoring features near the distal end of device. Two or more splines 1010 can be spaced evenly around circumference of the distal tip 1000d.

As shown in FIG. 12B, the splines or wire detents 1010 can have ends 1012 which can be pointed, rounded, have a ball end, and/or spiral swirled end. The wires can be pushed forward to grip in.

As shown in FIGS. 12C and 12D, the splines 1010 can be facing forward and/or backward from the direction of device 1000 entry or insertion. The spline features can be combined with a retractable needle tip feature 1020 where the device is anchored in place and then the needle is advanced into the tumor. The spline features can enter into healthy tissue, tumor tissue, or both. For example, the device 1000 can be deployed by inserting a bullet nose 1040 of the device 1000 into the tumor, advancing the splines 1010 (e.g., barbs), and advancing the retractable needle tip 1020 into the tumor. Anchoring before advancing the needle tip 1020 is advantageous if the tumor is difficult to pierce.

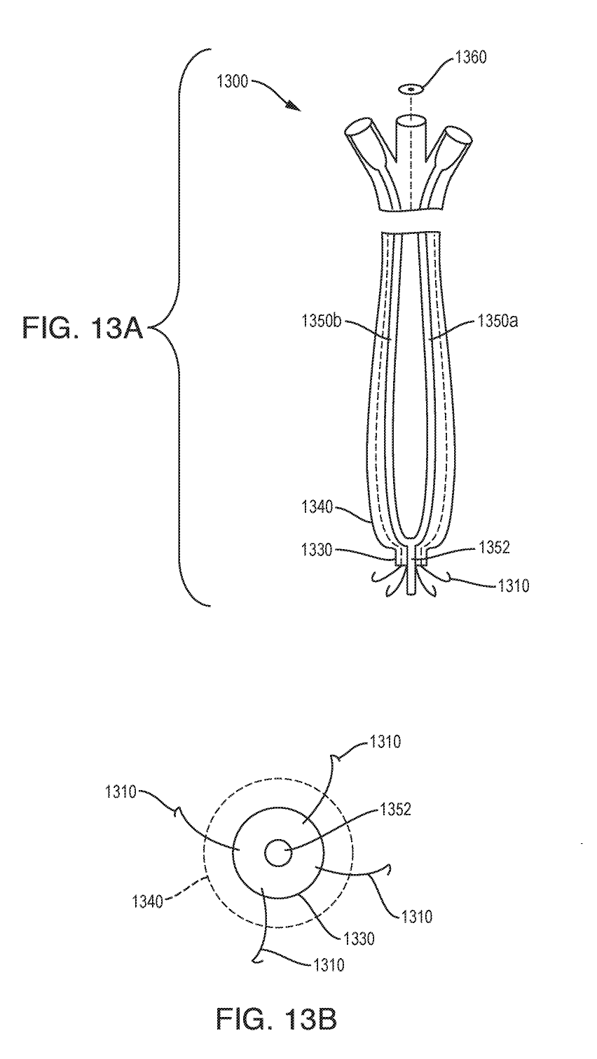

As shown in FIGS. 13A and 13B, retractable splines can include hook features 1310 for anchoring to tissue. Retractable splines can be controlled with a feature at the proximal end of the device 1300 such as an anchoring hook control 1360. The anchoring hook control 1360 can be a push-pull mechanism or a screw mechanism. Spline control can be separate from the fluid channel proximal interface features. The device 1300 can feature multiple independent lumens 1350a and 1350b (collectively 1350) running through the body of the device. The lumens 1350 can combine into a single lumen 1352 at the end of the device 1300. The device 1300 can be either rigid or flexible. The body of the device 1300 can include a overtube/step 1330 and a bullet nose 1340.

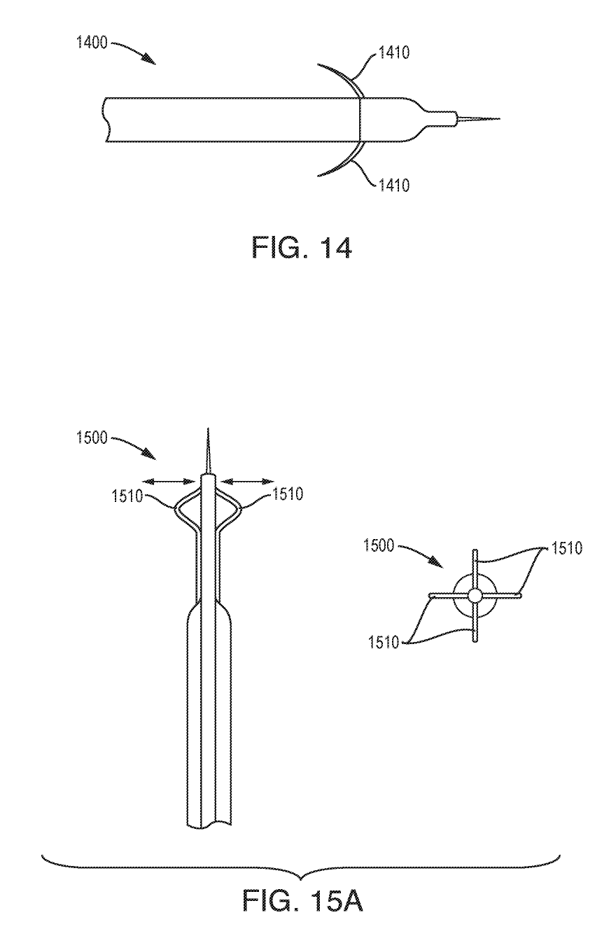

As shown in FIG. 14, the device 1400 can include an anchoring feature in the form of twines 1410 that are configured to pop out from the body of the device.

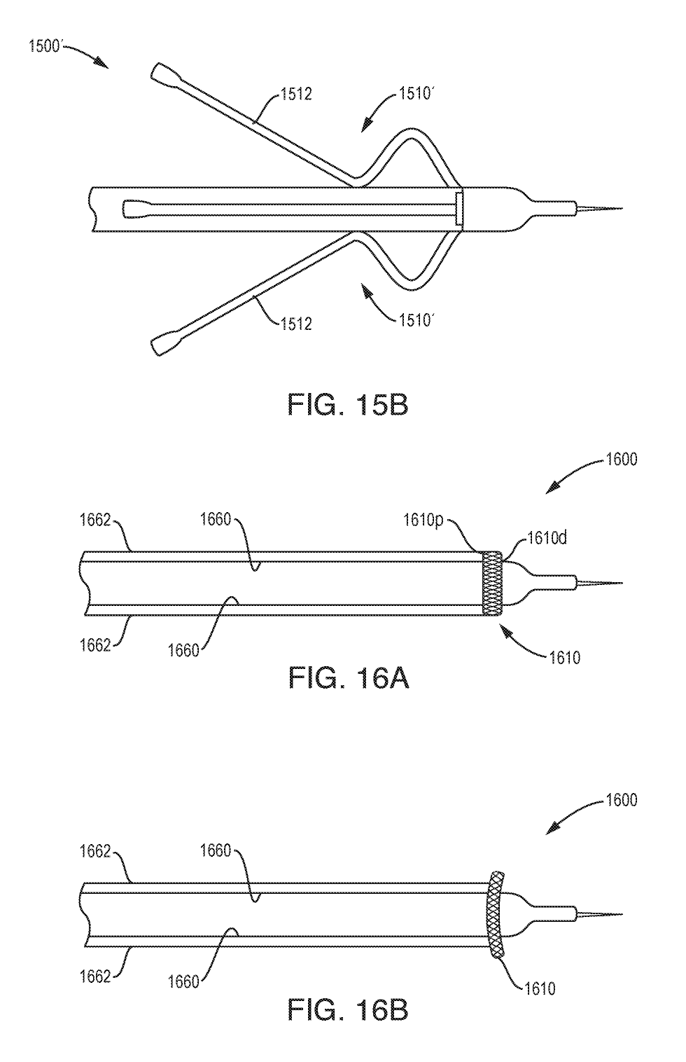

As shown in FIG. 15A, the device 1500 can include an anchoring feature in the form of a retractable basket-spline 1510 which expands around the outside diameter (OD) of the device and is fixed to the device on both ends. The device 1500 can include any number of separate splines 1510, e.g., two or more.

As shown in FIG. 15B, the device 1500′ can include a variation of the anchoring feature shown in FIG. 15A, in which the basket splines 1510′ also have legs 1512 which expand and create additional engagement with the surrounding tissue.

As shown in FIGS. 16A and 16B, the device 1600 can include an anchoring feature in the form of a mesh basket 1610 that is attached at the distal end 1610d to an inner tube 1660 and at the proximal end 1610p to an outer tube 1662. The basket 1610 can rest tight to the outer diameter of the part during insertion. The mesh basket 1610 can be created with a braid or winding technique. When anchoring is desired, a tube 1660 or 1662 or other feature can be extended, causing the distance between the proximal and distal ends 1610p, 1610d of the mesh basket 1610 to decrease and the outer diameter of the mesh basket to expand, engaging surrounding tissue. In some embodiments, the mesh basket 610 can be opened and closed by twirling or swirling one of the tubes relative to the other tube. In some embodiments, the mesh basket can be replaced with a flexible balloon or polymer doughnut.

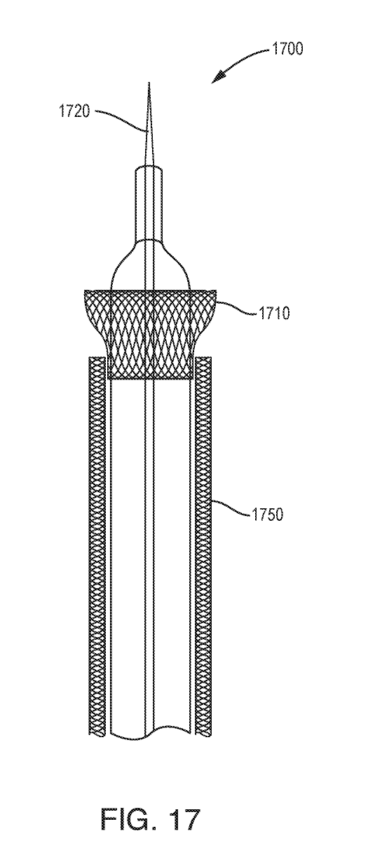

As shown in FIG. 17, the device 1700 can include an anchoring feature in the form of a stent style self-expanding scaffold 1710 made with Nitinol or other shape memory material. The stent material can be bonded at one end to the device 1700. A sheath 1750 can be extended and retracted over the stent to collapse the scaffolding tissue anchor feature 1710. Advancing the sheath or retracting the needle 1720 into the sheath can collapse the anchor 1710. The anchoring scaffold 1710 can be deployed by withdrawing the sheath 1750 with the needle 1720 in position.

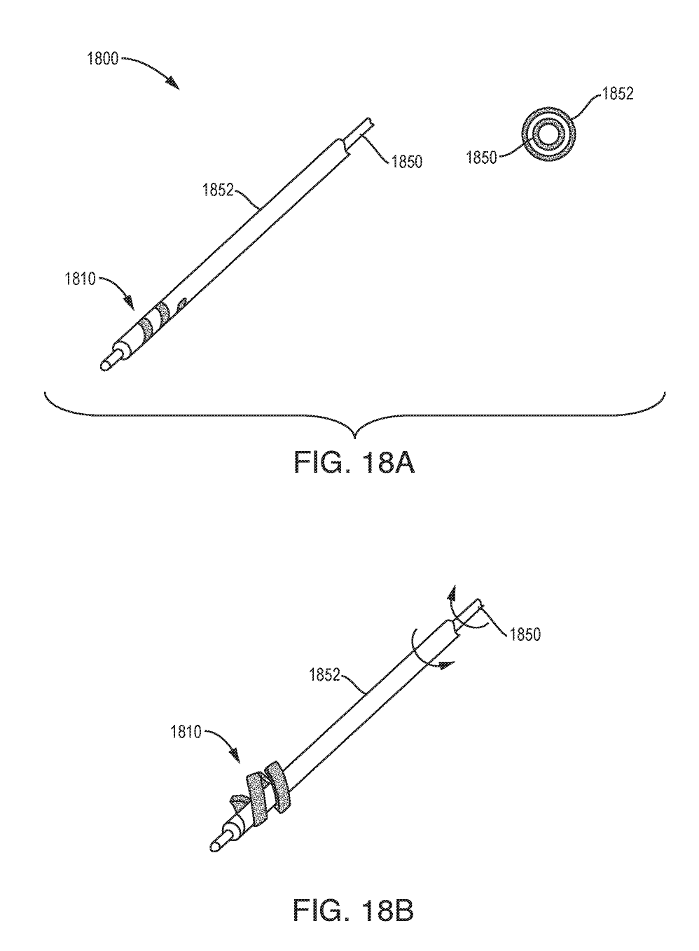

As shown in FIGS. 18A and 18B, the device 1800 can include an anchoring feature in the form of an expandable snare 1810. The snare 1810 can be helical. The snare/helix 1810 can be attached at the distal end to an inner tube 1850 and at the proximal end to an outer tube 1852. Alternatively, the snare/helix 1810 can be cut into the outer tube 1852 and attached to the inner tube 1850 at a location distal to the snare/helix. When the outer tube 1852 is rotated relative to the inner tube 1850 (e.g., twisted in opposite directions), the snare/helix 1810 can expand in outside diameter to engage the surrounding tissue.

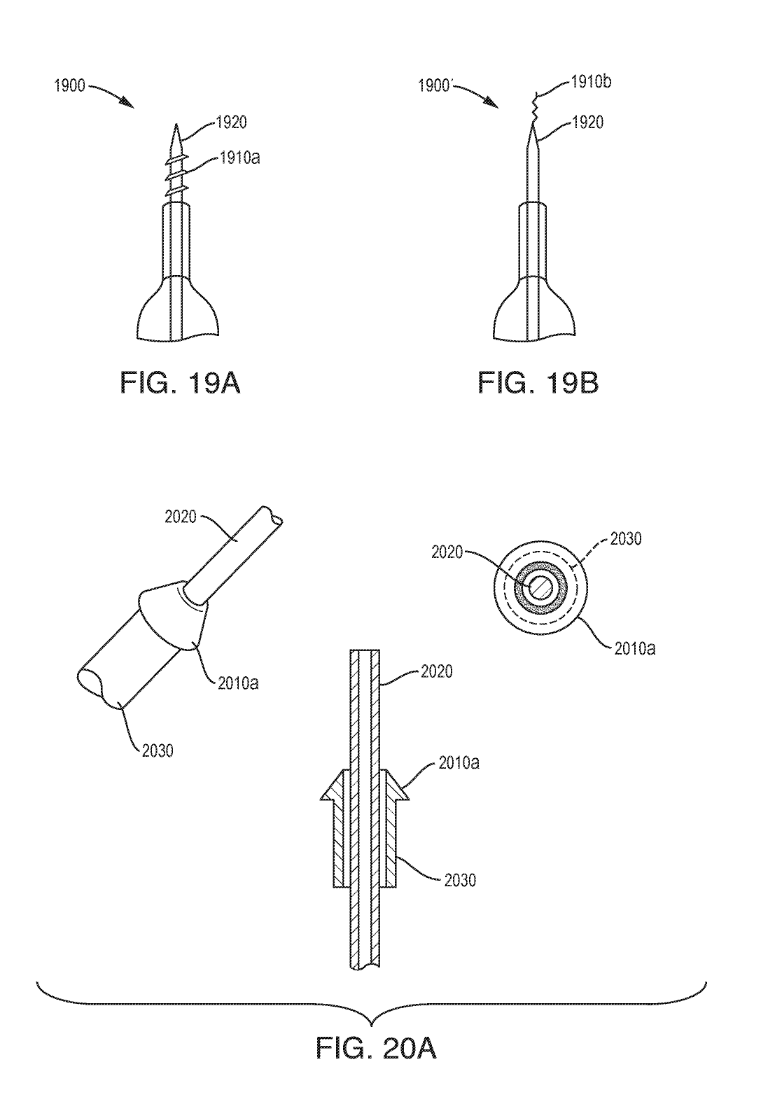

As shown in FIG. 19A, the device 1900 can include an anchoring feature in the form of a threaded barb 1910a. The barb 1910a can be formed on the outside diameter of the distal tip 1920 of the device 1900. The barb 1910a can be threaded or screwed into tissue. The threaded feature can be a retractable helix that can be screwed distally or unscrewed proximally. As shown in FIG. 19B, the anchoring feature can be in the form of a screw feature 1910b attached to the distal tip 1920 of the device 1900′ and extending distally in a corkscrew manner.

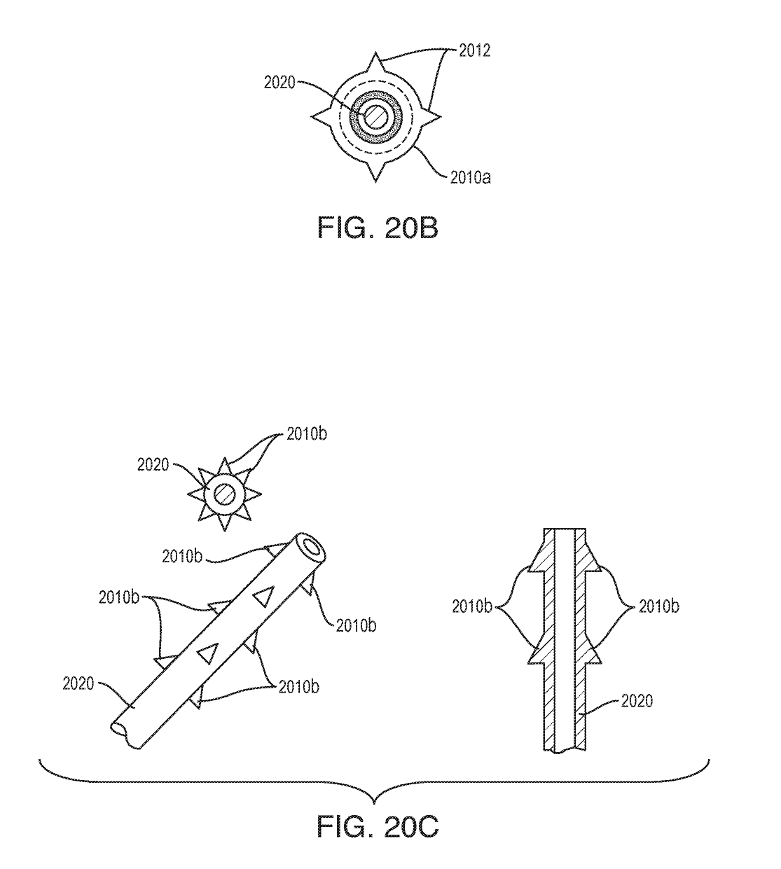

As shown in FIGS. 20A-20C, the anchoring feature can be in the form of barb features incorporated into the outer tube 2030 or microtip 2020 in a variety of locations and positions. As shown in FIG. 20A, a full diameter barb feature 2010a can be formed from the original tube material 2030 or an additional component added to the distal end of the outer tube 2030. The barb tip can anchor into tumor tissue and may provide a sealing barrier to prevent backflow. As shown in FIG. 20B, the barb feature 2010a can include one or more tangs 2012.

As shown in FIG. 20C, the barb features 2010b on the needle tube 2020 are configured to anchor into a tumor. Various arrays of barbs 2010b can be arranged at different locations and positions. The barb features 2010b can be formed using a variety of processes including cold form coining process on the solid wall thickness of the hypodermic tubing and then machined or laser cut to generate the final barb profile. The barb features 2010b can form a continuous outer surface or can contain one or more openings. The openings can be in the fluid path and can allow infusate to exit through them.

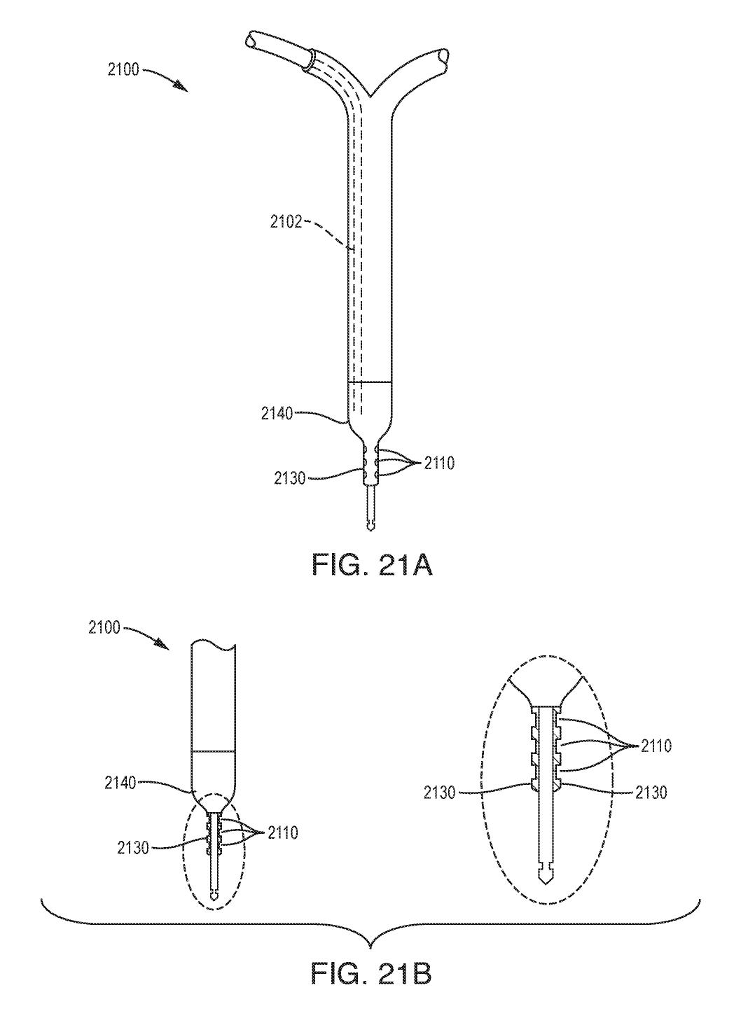

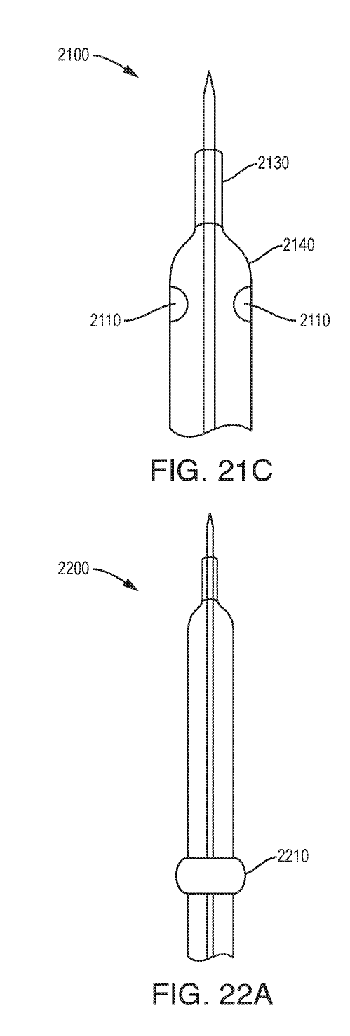

As shown in FIGS. 21A and 21B, the device 2100 can include an anchoring feature in the form of suction between the device and the tissue. The device 2100 can include one or more suction openings 2110, e.g., disposed in or on an over-tube 2130 of the device 2100. The suction openings 2110 can be in communication with a vacuum source (not shown) via a separate fluid lumen 2102. As shown in FIG. 21C, the suction openings 2110 can be formed on a main body of the device 2100, e.g., a bullet nose body or tube 2040.

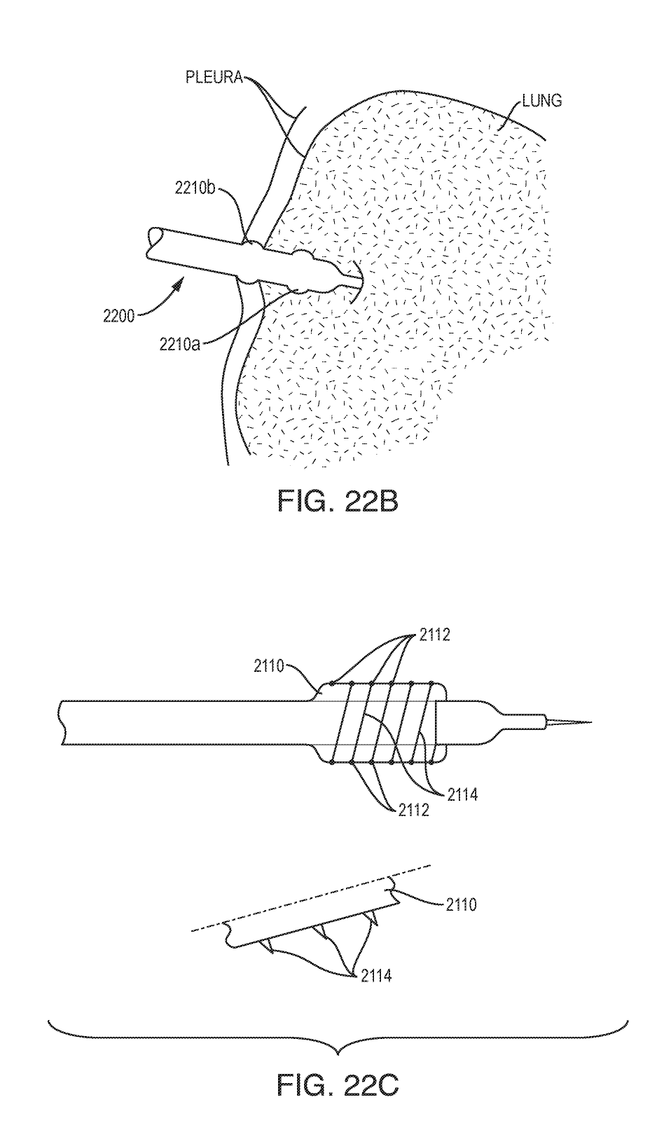

As shown in FIG. 22A, the device 2100 can include an anchoring feature in the form of a balloon 2110 that can be expanded between the device and the surrounding tissue, e.g., intercostal muscle tissue. As shown in FIG. 22B, the balloon can be located in either the intra-pleural space, or at a location in or near the tumor. For example, a first balloon 2110a can be deployed in the tumor and a second balloon can be deployed at the pleura cavity 2110b The balloon 2110a can augment the outside diameter of the device in the bullet nose area to additionally prevent backflow of therapy during delivery. As shown in FIG. 22C, the balloon 2110 can include gripping features on its surface to aid in tissue engagement. Gripping features can include a textured surface, gripping dots 2112, fingers 2114 which are extended when the balloon is expanded, and so forth.

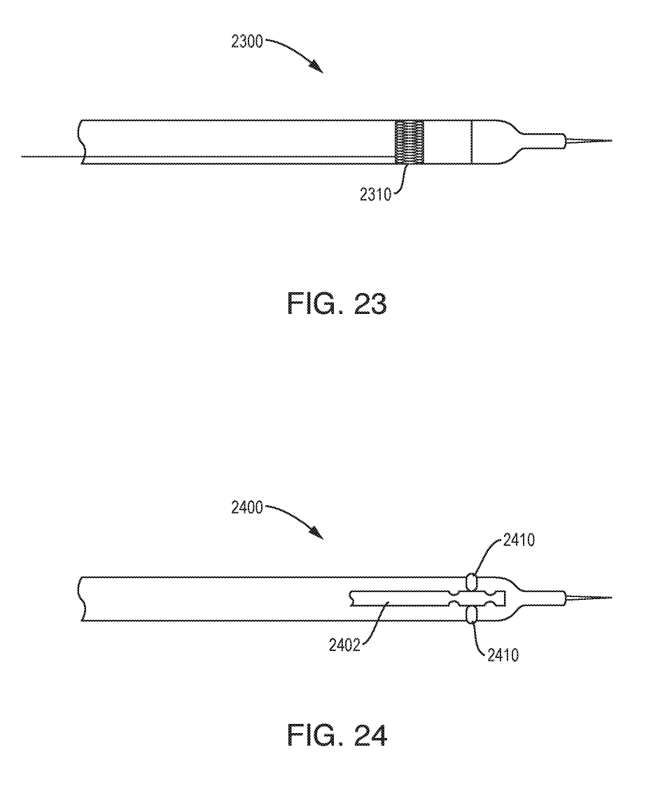

As shown in FIG. 23, the device 2300 can include an anchoring feature 2310 that utilizes heat and/or current to attach the device to the tissue, e.g., by ablating, cauterizing, melting, or otherwise modifying the tissue to cause it to stick to or grip the device.

As shown in FIG. 24, the device 2400 can include an anchoring feature in the form of one or more balls 2410 that are engaged and pushed outside the body outside diameter to engage tissue. An inner wire 2402 with indents or grooves can be used (e.g., pulled or pushed) to extend and disengage/retract the ball features 2410 outside the body surface.

As shown in FIG. 25A, the device 2500 can be rigid and can be used to access a tumor percutaneously. The device 2500 can be used in conjunction with a rigid insertion tube 2550 to aid in navigation to the tumor location. As shown in FIG. 25B, the device 2500 can be locked to the insertion tube 2550, e.g., with a Tuohy Borst mechanism. The outer tube/insertion tube 2550 can have a fixation mechanism (e.g., suction, wire hooks, balloons, etc.). The insertion tube 2550 locks the infusion needle device 2500 so that the device moves with the fixation device.

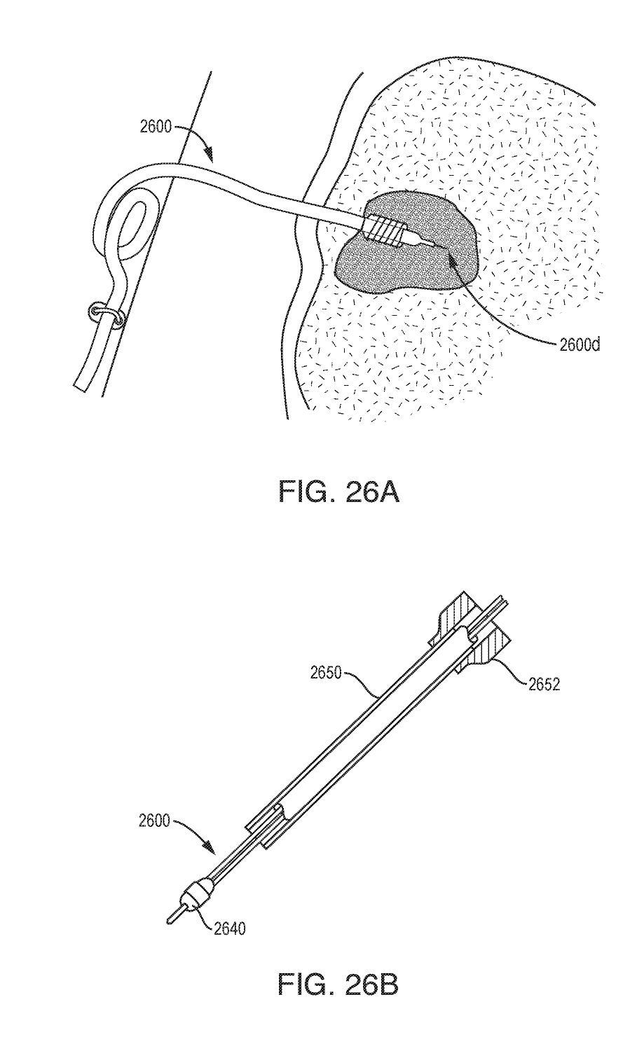

As shown in FIG. 26A, the device 2600 can be flexible and can be used to access a tumor percutaneously. The device 2600 can be anchored at both the tumor location and outside the skin, at only one of these points, or at any of a variety of other locations. The device 2600 can include slack in the length of the device which can aid in reducing movement at the distal device tip 2600d during respiration.

As shown in FIG. 26B, a stiff insertion tube 2650 can be used to navigate the device 2600 to the tumor or other target location. The bullet nose 2640 of the device 2600 can be the same outside diameter as the insertion tube 2650, and the device tip can lead the way navigating to the tumor. For example, a stiff outer sheath of the insertion tube 2650 can push the bullet nose tip 2640. Once the device 2600 is in the location of the tumor, the rigid insertion tube 2650 can be retracted or removed, exposing a flexible catheter connected to the bullet nose 2640. The rigid insertion tube 2650 can be a break away or butterfly sheath design. The rigid insertion tube 2650 can be a standard cannula, or can have various other configurations. A proximal end of the insertion tube 2650 can have a hub or handle 2652. The lumens of the device 2600 can be layered with additional materials for control flexibility, torsional rigidity, and/or other properties.

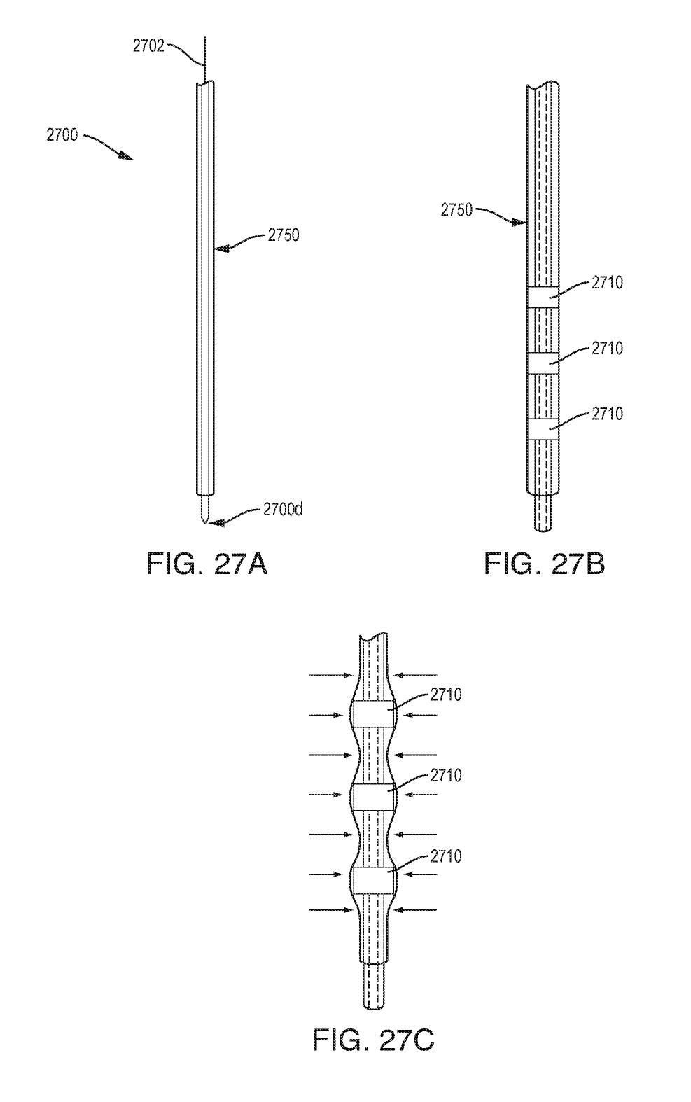

As shown in FIGS. 27A, 27B, and 27C, the device 2700 can be kept rigid during insertion and can be anchored to the patient. A core needle wire 2702 in the inside diameter of the device 2700 can be retracted when the device is at the target location. The device 2700 can include rigid features 2710 near the distal tip of the device 2700d, such as marker bands, rings, and spiral structures. Patient tissue can be in contact with the outside of the device 2700. The outer device jacket 2750 can be soft and can collapse to a smaller outside diameter when the core needle 2702 is removed. Since the rigid features 2710 near the tip do not collapse, this can create a varying outside diameter near the distal tip 2700d that engages the tissue to anchor the device.

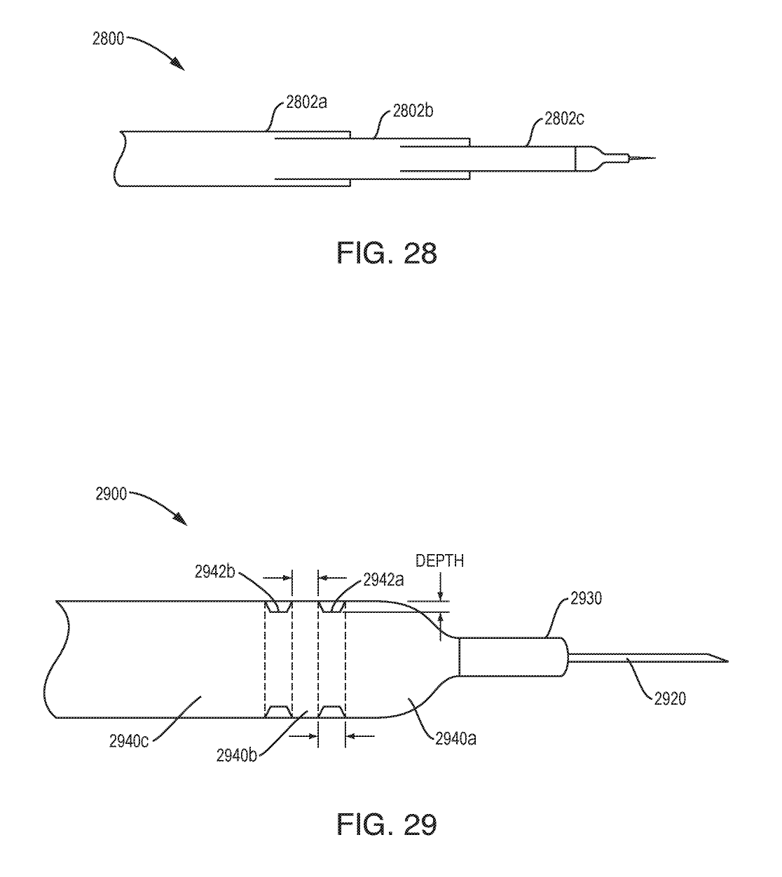

As shown in FIG. 28, the device 2800 can include two or more telescoping tubes, e.g., 2802a, 2802b, and 2802c (collectively 2802).

As shown in FIG. 29, the device 2900 can include one or more secondary grooved bullet nose features 2940b and 2940c proximal to the primary and most distal bullet nose 2940a. Compliant tissue can seal against the outside diameter of the device 2900, and the grooves 2942a and 2942b proximal to the primary bullet nose 2940a can assist in keeping that seal to prevent excessive backflow. Secondary grooved bullet nose features 2940b can have a specified pattern, depth, width, and shape to seal the device in the tissue. The over-tube 2930 and the microtip 2920 can be withdrawn during insertion to protect the device and tissue and can be extended for infusion. The secondary bullet nose grooves can be radiopaque for visualization under computed tomography (CT) or other imaging techniques.

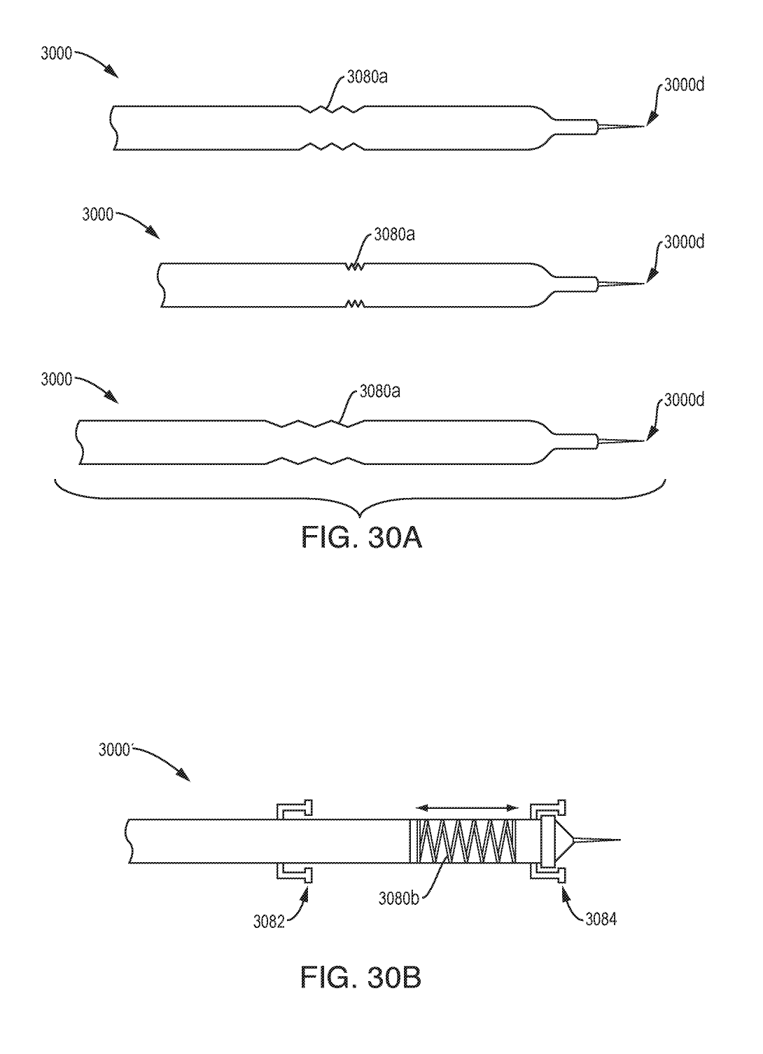

As shown in FIG. 30A, the device 3000 can include features that allow it to be flexible and to not tug on or otherwise move the distal tip during respiration. For example, the device 3000 can include an accordion type feature 3080a in the device body. Allowing the device 3000 to flex during respiration without causing the distal tip 3000d to move can assist in maintaining the seal of the tissue around the tip and therefore prevent backflow during infusion. For example, the accordion type feature 3080a can be put in tight and then pulled back to achieve flexibility.

As shown in FIG. 30B, the device 3000′ can include a spring feature 3080b that extends and contracts with lung movement during respiration. The device 3000′ can be fixed at the chest or chest wall, at the pleura, at the tumor, and/or at other locations. As shown, the two fixation features 3082, 3084 can secure the device 3000′ in place. The first fixation feature 3082 can attach to the outer chest wall and the second fixation feature 3084 can attach to the pleura or tumor. The spring 3080b allows the tip 3000d of the device 3000′ to move in conjunction with the tumor.

FIG. 31 illustrates an exemplary embodiment of a percutaneous lung intratumoral therapy delivery device 3100. The device 3100 can include one or more fluid lumens through which fluid can be delivered to a target site within a patient, and/or through which fluid or other material can be extracted from a target site within a patient. The device 3100 can include a distal tip 3120 having a fluid port 3125 therein. The tip 3120 can be a microfabricated structure, as described in U.S. Pat. No. 8,992,458 referenced above and incorporated herein by reference. The tip 3120 can be a single-lumen tube. The device 3100 can include an over-tube 3130 disposed over the tip 3120 to define a tissue-receiving space between the outer surface of the tip and the inner surface of the over-tube. Tissue can be pinched, captured, or otherwise disposed within or across the tissue-receiving space to form a seal with the device, limiting or preventing proximal backflow of infusate. Over-tube features 3130 are described in U.S. Pat. No. 8,992,458 referenced above and incorporated herein by reference. The device 3100 can include one or more bullet nose features, as described in U.S. Pat. No. 8,992,458 referenced above and incorporated herein by reference. As shown in FIG. 31, the device 3100 can include a plurality of bullet nose features 3140a, 3140b, 3140c and 3140d (collectively 3140), each having a conical, curved, or otherwise tapered distal-facing surface. Each bullet nose 3140 can have the same or substantially the same maximum outside diameter. In other arrangements, one or more of the bullet nose features 3140 can have maximum outside diameters that differ from others. The bullet nose features 3140 can be arranged in a spaced relationship along the length of the device 3100, proximal to the over-tube feature 3130. The bullet noses 3140 can define a ribbed or grooved section of the device against which tissue can be sealed to limit or prevent backflow. In some embodiments, tissue can be received within the void spaces defined between the adjacent bullet noses 3140 to form a tight seal. Fluid that could otherwise flow back proximally along the exterior of the device can be captured in the valleys defined between the successive bullet noses 3140. The plurality of bullet noses can be disposed in a spaced relationship along the length of the device to engage surrounding tissue as a means for anchoring the device and to limit or prevent backflow of infusate along the exterior of the device.

The device 3100 can include any of the anchoring features described herein. For example, as shown, the device 3100 can include a plurality of deployable splines 3110. The splines 3110 can be slidably mounted within longitudinal grooves or channels formed in the body of the device 3100. The splines 3110 can be flexible and/or resilient. The splines 3110 can have a heat-set shape. The splines 3110 can have a resting state in which they flare outward from the body, e.g., substantially 90 degrees from the body as shown. The device 3100 can include an actuator for controlling deployment and/or retraction of the splines. For example, a proximal handle of the device 3100 can include a collar that is rotatable or longitudinally slidable relative to the body of the device to actuate the splines. The splines 3110 can be pulled proximally in a longitudinal direction to retract the splines into grooves formed in the device 3100, flexing the splines away from their resting shape. The splines 3110 can be urged distally in a longitudinal direction to deploy the splines, e.g., by pushing them out of the grooves of the device 3100 and allowing them to return towards their resting shape. When deployed, the splines 3110 can engage with surrounding tissue to anchor the distal tip 3120 of the device 3100 thereto.

The body of the device 3100 can connect to a flexible or rigid catheter or tubing, which in turn can be coupled at a proximal end to a fluid source, pump, syringe, vacuum source, or the like.

The device 3100 can include an outer cannula or introducer sheath/delivery tube 3150. The device 3100 can be delivered through this tube 3150. In use, the cannula 3150 can be inserted percutaneously through the skin, muscle, pleura, etc. of the patient to access target anatomy, such as a pulmonary tumor. The cannula 3150 can be inserted with a stylet disposed therethrough. Once the distal tip 3150d of the cannula 3150 is close to the tumor, e.g., about 2 cm away, the stylet can be removed and the device can be inserted through the cannula. The cannula 3150 can help protect the relatively delicate device 3100 during insertion into the patient. The device 3100 can be advanced distally to position a distal tip 3120 or fluid port 3125 of the device within the tumor or in close proximity thereto. The anchoring feature of the device 3100 can be deployed to anchor the distal tip 3120 of the device in place, preventing movement of the device during respiration or other patient movement. A fluid, e.g., a drug or therapy containing fluid, can be delivered through the device and into the tumor.

An exemplary method of using the devices disclosed herein is as follows:

1. Imaging of location of tumor, access planning

2. Skin incision

3. Outer cannula and stylet advancement to tumor location using CT for guidance.

Stop approximately 2 cm from tumor

4. Remove stylet

5. Insert device through outer cannula, advance into tumor. May target approximate center of over-tube for center of tumor

6. Advance splines to anchor tip

7. Remove or leave in cannula. If removing cannula, can remove by splitting sheath or keep on device between the skin and the hub.

8. Infuse through device into tumor

9. Retract back splines

10. Remove device from cannula

11. Potential for injection of blood plug or bio gel as cannula is removed.

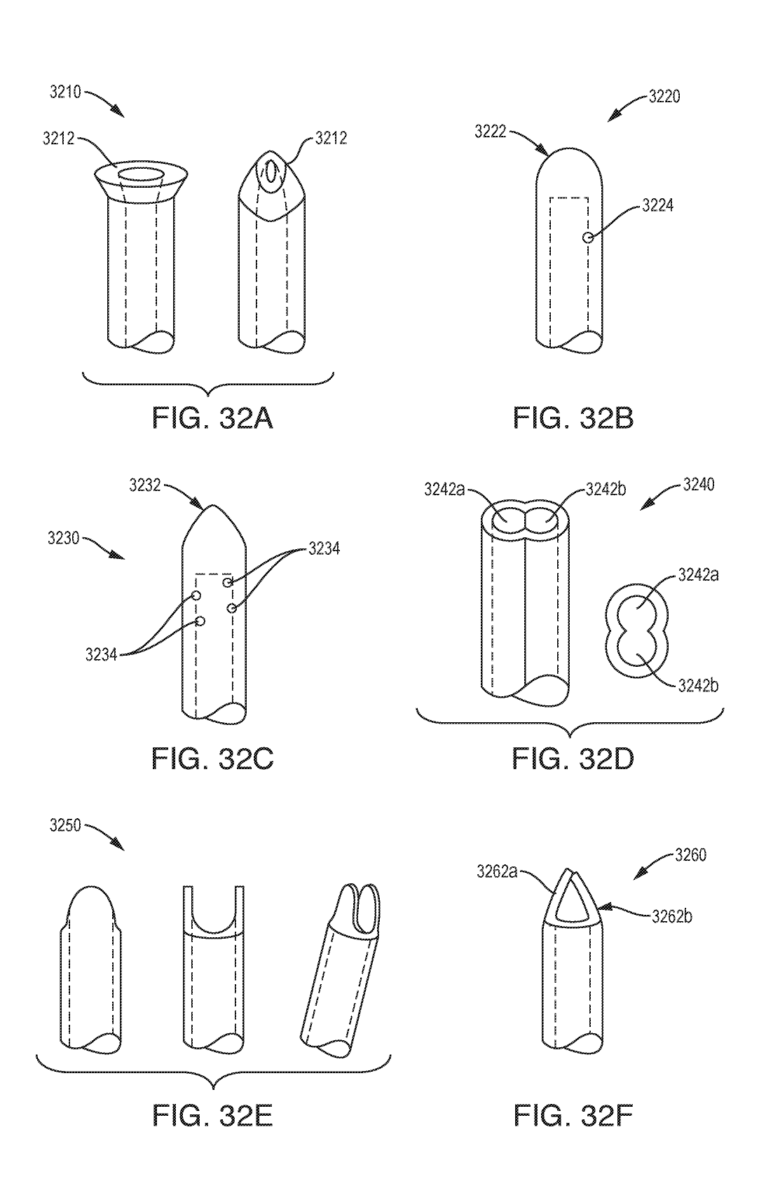

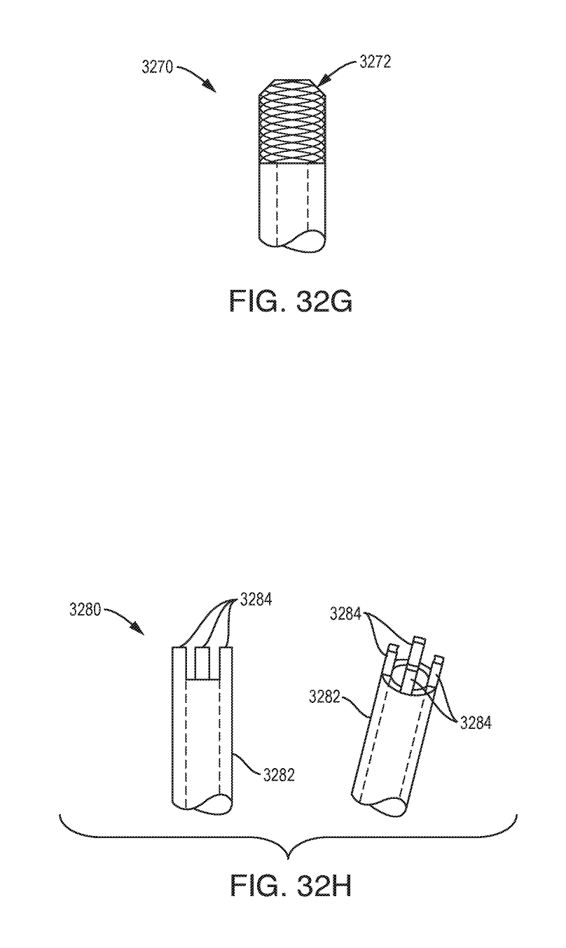

FIGS. 32A-32H illustrate exemplary needle tip geometries that can be used in any of the devices described herein, e.g., in the micro tip of the device. The illustrated geometries can be configured to minimize coring due to the needle profile, thereby facilitating consistent flow through the device. FIG. 32A illustrates a flattened tube geometry 3210 that may be less likely to core due to the asymmetrical lumen profile 3212. FIG. 32B illustrates a side port geometry 3220 in which the needle has a blunt tip 3222 with one or more side-facing fluid ports 3224. FIG. 32C illustrates a needle geometry 3230 with a conical blunt tip 3232 and an array of very small holes 3234 spaced around the outside diameter of the tip. The needle geometry of FIG. 32C is less likely to block due to the radial flow of fluid. FIG. 32D illustrates a “FIG. 8” tip geometry 3240 in which two inner lumens 3242a and 3242b are joined at one side to form a larger lumen having a transverse cross-section generally in the shape of the number eight. FIG. 32E illustrates an “open duck bill” tip configuration 3250. FIG. 32F illustrates a “closed duck bill” tip configuration 3260 having tabs 3262a and 3262b bent over from the “open duck bill” tip configuration. FIG. 32G illustrates a needle 3270 having a breather vent tip 3272. The breather vent tip 3272 can be a very fine, e.g., less than 5 um, filter attached to the needle tip. The needle 3270 of FIG. 31G is less likely to block due to the radial flow of fluid. FIG. 32H illustrates a tip having a “rook” or “castle” geometry 3280. The tip 3282 can include any number of prongs 3284, e.g., 4 or more prongs at the end of the tip. The prongs 3284 can be sharpened to improve penetration.

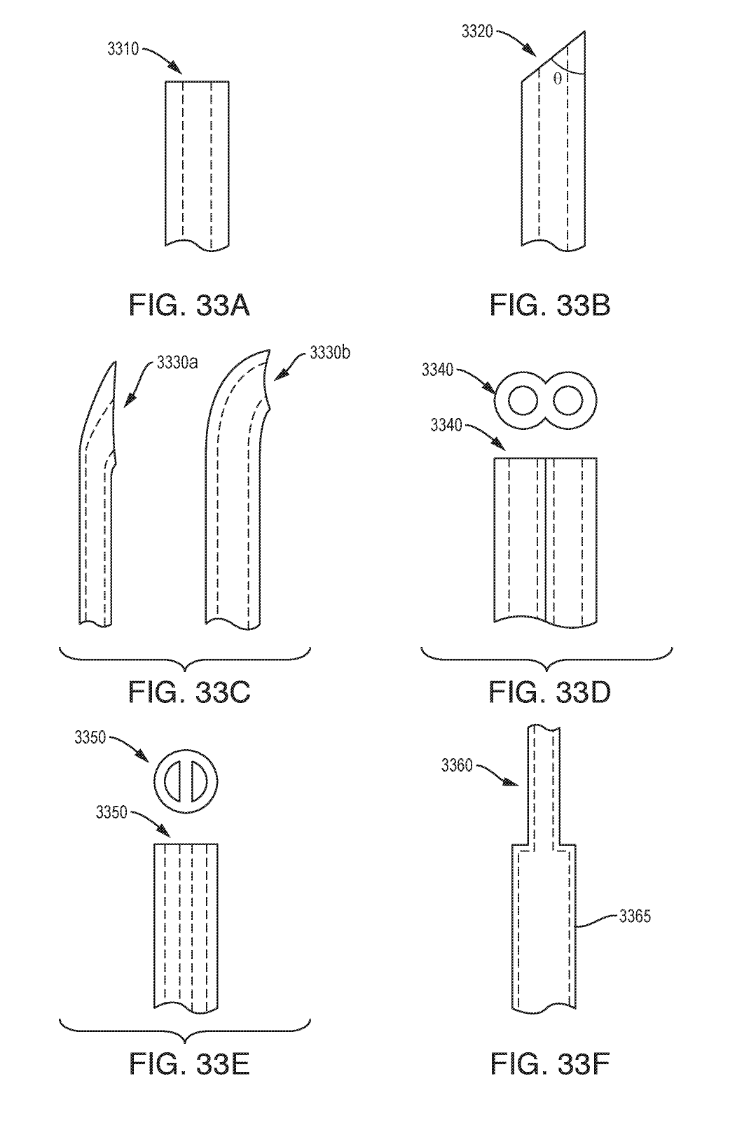

FIG. 33A-33F illustrates additional examples of needle tip geometries that can be used in any of the devices described herein, e.g., in the micro tip of the device. As shown in FIG. 33A, the needle tip can be a blunt tip 3310. As shown in FIG. 33B, the needle tip can be a beveled tip 3320 having multiple angle options θ. As shown in FIG. 33C, the needle tip can be a non-coring tip, e.g., 3330a and/or 3330b. As shown in FIG. 33D, the needle tip can be a double tip 3340. As shown in FIG. 33E, the needle tip can be a dual lumen or DD tip 3352. As shown in FIG. 33F, the needle tip 3360 can be combined with an overtube feature 3365.

Devices are disclosed herein having an anchoring feature that can allow the distal tip of the device to remain in a substantially fixed location relative to a target location, e.g., a patient's tumor, during patient movement, such as during respiration. The devices herein can be used in any part of the body that moves during infusion.

Devices are disclosed herein having a seal feature for limiting or preventing backflow of infusate along the exterior of the device.

Although the invention has been described by reference to specific embodiments, it should be understood that numerous changes may be made within the spirit and scope of the inventive concepts described. Accordingly, it is intended that the invention not be limited to the described embodiments.

1. A drug delivery device, comprising:
a distal tip having one or more fluid ports therein;
an inner fluid lumen configured to convey fluid to the one or more fluid ports of the tip; and
a plurality of bullet noses disposed in a spaced relationship along a length of the device proximal to the distal tip.

2. The device of claim 1 wherein the plurality of bullet noses are configured to limit or prevent backflow of infusate along an exterior of the device.

3. The device of claim 1, further comprising means for anchoring the distal tip to target tissue of a patient to prevent movement of the distal tip relative to the target tissue during patient movement, wherein the means for anchoring is separate from the plurality of bullet noses.

4. The device of claim 3, wherein the target tissue of a patient comprises a tumor.

5. The device of claim 3, wherein the patient movement comprises respiration.

6. The device of claim 1, wherein the device includes one or more over-tubes disposed over the distal tip to define a tissue-receiving space.

7. The device of claim 6, wherein tissue is received within the tissue-receiving space to limit or prevent backflow of infusate along an exterior of the device.

8. The device of claim 1, wherein one or more of the plurality of bullet noses have a conical, curved, or tapered exterior surface.

9. The device of claim 1, wherein the plurality of bullet noses engage surrounding tissue to anchor the device.

10. The device of claim 3, wherein the means for anchoring comprises one or more splines deployable from an exterior of the device to engage surrounding tissue.

11. The device of claim 3, wherein the means for anchoring comprises one or more balloons deployable from an exterior of the device to engage surrounding tissue.
